(19) United States
(12) Patent Application Publication
(10) Pub. No.: US 2026/0215946 A1
(43) Pub. Date: Jul. 30, 2026

(54) ANTEGRADE URETHRAL ENDOSCOPE

(71) Applicant: BUTTERFLY MEDICAL LTD., Yokneam (IL)

(72) Inventor: Idan Geva, Even Yehuda (IL)

(73) Assignee: BUTTERFLY MEDICAL LTD., Yokneam (IL)

(21) Appl. No.: 19/153,326

(22) PCT Filed: Feb. 1, 2024
(86) PCT No.: PCT/IL2024/050129

§ 371 Date: Aug. 3, 2025

Related U.S. Application Data
(60) Provisional application No. 63/443,399, filed on Feb. 5, 2023.

(51) Int. Cl. A61F 2/966; A61B 1/00; A61B 1/018; A61B 1/307; A61F 2/04

(52) U.S. Cl. CPC A61F 2/966; A61B 1/00154; A61B 1/018; A61B 1/307; A61F 2002/047

(57) ABSTRACT

A urethral endoscope for antegrade insertion of a stent is disclosed. The urethral endoscope and method may provide opties at the time of inserting and positioning a urethral stent. A deployment system is configured to hold simultaneously an implant and an optical system facilitating direct insertion of the implant into the desired area. In some embodiments, this facilitates insertion of the implant without first inserting the implant into the bladder and/or pulling the implant back from the bladder into the urethra. Some embodiments may facilitate insertion of an implant with a reduced number of insertions and removals of the endoscope and/or a sheath thereof.

### RELATED APPLICATIONS

This application is a Continuation in Part of International patent application PCT/IL2024/050129 filed on 1 Feb. 2024 which claims the benefit of priority under 35 USC § 119(e) of U.S. Provisional Patent Application No. 63/443,399 filed 5 Feb. 2023, the contents of which are incorporated herein by reference in their entirety.

### FIELD AND BACKGROUND OF THE INVENTION

The present invention in some embodiments thereof relates to a urethral endoscope for insertion of an implant (e.g., a stent) and, more particularly, but not exclusively, to a urethral endoscope and method which can provide antegrade inserting and positioning a urethral stent.

U.S. Pat. No. 11,571,290 to the present applicant appears to disclose that, “Embodiments of a Urological implant include an implant with an elongated body having a longitudinal axis. Optionally longitudinal ribs symmetrically oppose each other and are connected to elongated body. The longitudinal ribs are optionally elastically shiftable between a collapsed state and an expanded state relative to the spinal longitudinal axis, in order to retract or/and support periurethral tissue. Optionally the system includes an implant extraction handle. The extraction handle is optionally positioned proximally to the elongated body and connected to the longitudinal rib and subject to a pulling force to facilitate and/or force approximation of the longitudinal supports to the longitudinal axis. In some embodiments, an implant body includes longitudinally spaced arched members, interconnected via arch ends sequentially along a length of a first and second longitudinal rib. Optionally the arched members are elastically bendable to facilitate elastic contractibility of the implant body under a transverse compressive force.”

U.S. Pat. No. 10,507,122 to the present applicant appears to disclose a, “Urological (prostatic) implant, system, and method for retracting or/and supporting periurethral tissue enclosing a prostatic urethra along lengths of prostate lobes, for example, in BPH patients. Prostatic implant includes independently actuatable distal retractor incorporating craniolateral corners and proximal retractor incorporating caudolateral corners. Retractors may be connected via elongated spine member, and formed as paired curved wing-liked structures having interconnecting members. May further include tissue support members extending between elongated edge members connecting retractors to spine member, and tissue support members extending between elongated edge members and spine member. Tissue support members are sized and configured for supporting portions of prostatic lateral lobes when spine member engages anterior interlobar groove extending between prostatic lateral lobes, and when elongated edge members engage posterolateral interlobar grooves. System and method include implant manipulator detachably connected to implant elongated edge members, for manipulating and forcing implant caudolateral corners into close proximity, for delivery into subject.”

U.S. Pat. No. 7,507,247 appears to disclose a, “A stent delivery system for the placement of non-expandable stents in the urethra is described. The delivery system is generally composed of a catheter with a stent mounted near the distal end of the catheter. The stent is held in place by a balloon or mechanical mechanism.”

U.S. Pat. No. 11,096,774 appears to disclose that, “Systems, devices, and methods are provided for the delivery of an implant into the prostatic urethra. Embodiments of delivery systems can include a delivery device for insertion into the patient and a proximal control device for use in controlling release of the implant from the delivery device.”

US Patent Publication no. 2012/0010645 appears to disclose, “A dilation device for an intrabody lumen, for example, a urethra partially occluded by an enlarged prostate in the form of a curved element configured to be implanted around the lumen and add resilience to the lumen, and a method for dilating such a lumen by implanting a dilation device around the outside of the lumen with at least part of the dilation device embedded in surrounding, thereby preventing clinically significant compression of the lumen. Also, an implantation device for such a dilation device in the form of a mounting device configured to carry the dilation device; and a releasably attached movable holder, with the mounting device and holder configured to deliver the dilation device though a working channel to a desired position in a lumen, and to implant the dilation device through a wall of the lumen to a position surrounding the lumen.”

U.S. Pat. No. 7,967,830 appears to disclose that, “A method and apparatus for introducing a first elongate medical device and short wire guide that are coupled together into a work site and remotely disconnecting them within the work site such that a secondary device comprising a catheter member can be introduced over the wire guide to the work site, and/or a second wire guide can be introduced to the work site via a passageway of the primary access device. A separating member may be provided to remotely separate the wire guide from the elongate medical device. In one example of the method, a wire guide and primary access device (e.g., a sphincterotome) is coupled to the wire guide and introduced via a duodenoscope into the biliary system. In another example of the method, a prosthesis, such as a valve or stent, is placed within the work site coupled to a wire guide which is remotely disconnected within the work site and a secondary device, such as a dilation balloon or second prosthesis, is introduced into the work site after the first delivery system is removed.”

International Patent Publication no. WO2021072258 appears to disclose that, “Devices and systems are disclosed for managing and/or treating body tissues obstructing a hollow body lumen, including the prostatic lobe tissues obstructing the urethra, for example conditions including benign prostatic hyperplasia (BPH), bladder outlet obstruction (BOO), benign prostatic obstruction (BPO) and associated lower urinary tract symptoms (LUTS). An implant is maintained in a constrained configuration within a distal portion of an elongated sheath so that is may be deployed by driving the implant with a pusher coaxially disposed within the sheath. An implant engaging element helps maintain control of the implant during deployment.”

U.S. Pat. No. 11,690,744 appears to disclose that, “A stent delivery system may include a delivery device and a tubular body having a lumen sized to slidably fit about an outer diameter of the delivery device and a drainage stent having an anchoring mechanism, wherein the delivery device includes a constraining member configured to engage an external portion of the tubular body at the proximal end of the delivery device. A method of delivering a stent may include inserting a tubular body into the port of an endoscope, inserting a stent delivery device and a stent having an anchoring mechanism into the tubular body, wherein the delivery device includes a constraining member configured to engage and retain the tubular body at the proximal end of the delivery device, advancing the drainage stent distally through the tubular body, sliding the tubular body proximally, and engaging an external portion of the tubular body with the constraining member.”

U.S. Pat. No. 9,907,686 appears to disclose that, “A system for implanting a prosthesis includes a prosthesis delivery device having a handle, an inner lumen, a nose cone, an intermediate lumen, a prosthesis sheath at the distal end of the intermediate lumen for holding therein a prosthesis to be implanted, and an outer lumen fixedly connected to the handle. The system can include a lumen control assembly operable to move the intermediate lumen with the prosthesis sheath between a retracted position and an extended position.”

### SUMMARY OF THE INVENTION

According to an aspect of some embodiments of the invention, there is provided a method of inserting urethral implant storing implant in distal portion of a lumen of a delivery sheath; inserting the distal portion of the sheath into a urethra; inserting optics through proximal portion of lumen to view out of a distal opening of lumen while implant remain in distal portion of lumen.

According to some embodiments of the invention, the method further includes retaining the implant on a retainer and retracting the sheath exposing the implant inside the urethra.

According to some embodiments of the invention, the retainer extends from a proximal opening of the sheath to the implant.

According to some embodiments of the invention, the retainer includes a tube and the method further including: grasping proximal portion of implant with loop thread passing through to a proximal opening of the tube.

According to some embodiments of the invention, the method further includes cutting one leg of the loop and stringing thread through tube to release the implant.

According to some embodiments of the invention, the grasping is on two proximal corners of implant.

According to some embodiments of the invention, implant expand upon the exposing.

According to some embodiments of the invention, the method further includes retracting the optics with the retracting of the sheath.

According to some embodiments of the invention, the method further includes advancing the optics after the exposing.

According to some embodiments of the invention, the advancing includes causing a distal portion of the optics to protrude from the distal opening of the lumen.

According to some embodiments of the invention, the method further includes re-extending the sheath after the retracting to return the implant into the distal opening of the lumen.

According to an aspect of some embodiments of the invention, there is provided system for inserting an implant into a urethra including: a sheath having a distal portion configured for insertion into the urethra while a proximal portion remains outside of the urethra a retainer configured for retaining the implant in a distal portion of the sheath a connector including a distal portion connecting the retainer and a handle configured to facilitate release of the connector.

According to some embodiments of the invention, the retainer extends through a lumen of the sheath from the implant to a proximal opening of the lumen.

According to some embodiments of the invention, the implant is retained in the sheath in a collapsed state.

According to some embodiments of the invention, the implant is configured to expand automatically upon exposure from the sheath.

According to some embodiments of the invention, the system further includes a retraction mechanism in the handle for retracting the sheath while the implant is retained in place by the retainer thereby exposing the implant in the urethra.

According to some embodiments of the invention, the retainer includes a tube and the connector includes a thread.

According to some embodiments of the invention, a loop of the thread passes out a distal end of the tube and is looped around the implant and wherein two free ends of the loop are exposed passing out a proximal opening of the tube.

According to some embodiments of the invention, the handle is configured to facilitate cutting one of the free ends and stringing the thread back out of the retainer from the proximal opening of the tube thereby releasing the implant.

According to some embodiments of the invention, the system further includes an optical assembly having a distal portion that fits through the sheath while the implant is retained in the distal portion and while the retainer and connector are affixed to the implant and the optical assembly having a proximal portion accessible from outside the sheath for viewing through a distal opening of the sheath.

According to some embodiments of the invention, the handle further includes a mechanism for re-extending the sheath to return the implant into the distal portion of the sheath after the exposing.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is an image of a delivery system with a catheter in an extended configuration in accordance with an embodiment of the current invention;

FIG. 2 is an image of a delivery system with a catheter in a partially extended configuration in accordance with an embodiment of the current invention;

FIG. 3 is an image of a proximal side of a delivery system in accordance with an embodiment of the current invention;

FIG. 4A is an image of a distal end of a delivery sheath and implant after retraction of the delivery sheath in accordance with an embodiment of the current invention;

FIG. 4B is an image of a distal end of a delivery sheath and implant after retraction of the delivery sheath in accordance with an embodiment of the current invention;

FIG. 5 is an image of a handle of an implant delivery system in accordance with an embodiment of the current invention;

FIG. 6A is schematic illustration of a delivery system with the sheath in an extended configuration in accordance with an embodiment of the current invention;

FIG. 6B is schematic illustrations of a trolley and optical assembly in accordance with an embodiment of the current invention;

FIGS. 7A and 7B are distant and close-up cross-sectional views of a delivery sheath in accordance with an embodiment of the current invention;

FIG. 8 is a schematic diagram of the relevant areas of the urinary system which are relevant to the medical procedures that are relevant to some embodiments of the current invention;

FIG. 9 is a block diagram of an inserter system in accordance with an embodiment of the current invention;

FIGS. 10A and 10B are flow chart illustrations of methods in accordance with embodiments of the current invention;

FIG. 11A illustrates a sheath in an extended configuration in accordance with an embodiment of the current invention;

FIG. 11B illustrates a sheath 102 in a retracted configuration in accordance with an embodiment of the current invention;

FIGS. 11C and 11D illustrate a sheath in a retracted configuration with the optical assembly in an advanced position in a general view and expanded view respectively in accordance with an embodiment of the current invention;

FIG. 12A illustrates connecting an implant to a retainer tube in accordance with an embodiment of the current invention;

FIG. 12B illustrates tightening an implant to a retainer tube in accordance with an embodiment of the current invention;

FIGS. 12C and 12D illustrate anchoring a connector at a handle in accordance with an embodiment of the current invention; and

FIG. 13 is a flow chart of a method of affixing an implant to a delivery system in accordance with an embodiment of the current invention.

FIG. 14A is schematic illustration of a delivery system with the sheath in an extended configuration in accordance with an embodiment of the current invention.

FIG. 14B is schematic illustration of a delivery system with the sheath shown as transparent in an extended configuration in accordance with an embodiment of the current invention.

FIGS. 14C and 14D are close-up view of an optical assembly and retainer in a delivery system in an extended configuration in accordance with an embodiment of the current invention.

FIG. 15A is schematic illustration of a delivery system with the sheath in a retracted configuration in accordance with an embodiment of the current invention.

FIG. 15B is schematic illustration of a delivery system with the sheath shown as transparent in the retracted configuration in accordance with an embodiment of the current invention.

FIG. 16A is schematic illustration of a delivery system with the sheath in a retracted configuration in accordance with an embodiment of the current invention.

FIG. 16B is schematic illustration of a delivery system with the sheath shown as transparent in the retracted configuration in accordance with an embodiment of the current invention.

FIGS. 17A-17C are schematic illustrations of a handle of a deployment system including a release module for an implant in accordance with an embodiment of the current invention.

FIGS. 18A and 18B are schematic illustrations of a spool type tensioning element in accordance with an embodiment of the current invention

### DESCRIPTION OF EMBODIMENTS OF THE INVENTION

### Overview

The present invention in some embodiments thereof relates to a urethral endoscope for antegrade insertion of a stent and, more particularly, but not exclusively, to a urethral endoscope and method which can provide opties at the time of inserting and positioning a urethral stent,

Benign prostatic hyperplasia (BPH) (prostate gland enlargement) is a common condition in the aging male population. Among the treatments for BHP is for the doctor to insert a stent into the patient's urethra using an endoscope.

Present technique as often practiced inserts a urethral stent using what is called retrograde insertion. In this technique the endoscope is first inserted into the bladder. Then the stent is passed through the endoscope into the bladder. The endoscope is then removed. After this an endoscope equipped with a video optics system is reinserted and, under visual observation, the stent is pulled back from the bladder to the urethra.

A problem of this procedure is that the traditional endoscope generally does not pass the stent and hold video optics at the same time. This may result in the procedure including numerous steps. For example, retrograde insertion may include a preliminary insertion of the optic endoscope to measure the prostate. After the preliminary insertion, the endoscope may be removed, and a second insertion may be performed to place the stent in the bladder (e.g., a blind insertion, without optics). The scope may then be removed a second time, and the optic endoscope inserted again in order to view the positioning of the stent in the prostatic urethra as the stent is pulled back from the bladder into the urethra. Inserting and removing the endoscope multiple times, as well as inserting the stent into and out of the bladder, may increase the time required for the surgery, increase the recovery time, and/or increase the risk of complications.

In some embodiments of the current invention, a deployment system is configured to hold simultaneously an implant and an optical system facilitating direct insertion of the implant into the desired area. In some embodiments, this facilitates insertion of the implant without first inserting the implant into the bladder and/or pulling the implant back from the bladder into the urethra. Some embodiments may facilitate insertion of an implant without multiple insertions and removals of the endoscope and/or a sheath thereof, which may be inserted and/or removed fewer times (e.g., once or twice).

An aspect of some embodiments of the current invention relates to system for insertion of a urethral implant. Optionally, the system includes a sheath configured to insertion into the urethra (e.g., the distal end of the sheath is inserted into the urethra while the proximal portion remains outside the urethra) having a lumen used to pass optics and/or a retainer of the implant and/or other objects (e.g., fluids, tools) into the urethra. The implant may be retained at a distal end and/or in a distal portion of the sheath e.g., the distal ½ and/or the distal ¼ and/or the distal ⅛ and/or the distal 1/16. For example, the implant may be held via a retainer which may include for example a thin hollow tube and/or thread threaded through the hollow tube. Optionally, the thread may be attached to a proximal corner of the implant. For example, a loop of suture thread may be looped around a proximal feature of the implant (e.g., around a wire of the implant and/or through a void in a closed form of the implant). Optionally, the thread holds the proximal corner to a distal end of the tube. Optionally, the free ends of the loop may pass through the tube from the distal opening thereof and/or out a proximal opening thereof. Optionally, the free ends of the loop may pass through the sheath from the distal portion thereof and/or out a proximal opening thereof. The implant may be held firmly in against a distal end of the tube under tension from the thread e.g., both ends of the thread may be connected to tension preserving anchors.

In some embodiments, thread tension is preserved by an anchor. For example, the anchor may hold a proximal portion of the thread and/or the anchor may be connected to the sheath (e.g., a proximal handle thereof) by a threaded tensioning anchor. Optionally, rotating the anchor or an anchor bolt in one direction tensions the thread and rotating it in another direction loosens the thread. Alternatively or additionally, a proximal portion of the thread may be held in tension by an elastic element. For example, the anchor may be attached to the handle and/or the sheath via a spring that preserves tension. Optionally, the tension may be adjusted by a threaded mechanism. Alternatively or additionally, a proximal portion of the thread may be wound around a spool. For example, tension may be preserved on the spool by an elastic element (e.g., a torsion spring). Alternatively or additionally, tension may be preserved on a spool by a ratchet mechanism.

In some embodiments, pulling the thread back out from the tube (e.g., form the proximal end thereof) releases the implant (e.g., at the distal end thereof inside the urethra). For example, a first end of the thread may be released from its anchor (e.g., by cutting the thread optionally proximal to the tube). Then the first end of the thread may be pulled through the tube. Optionally, cutting and/or removing the thread releases the implant e.g., pulling on the second end of the thread to draw the first end of the thread back out through the tube. Optionally, a second tube with a second suture holds a second location on the implant. For example, the implant may include two proximal corners and/or each tube may be connected to a respective one of the proximal corners. Optionally, the implant is released into the urethra by releasing both threads. For example, the implant may be released after it is positioned in its desired place. Holding the implant in two places with two retainers may facilitate control of the position and angle of the implant. In order to release the thread, it may be cut manually (for example: with a scissor, etc.) where it exits at the device. Alternatively or additionally, the device may be fitted with an internal cutter to cut the thread.

In some embodiments of the current invention, the insertion of the sheath with the implant retained thereto may be performed together with the endoscopic optical device. Alternatively or additionally, the insertion of the sheath with the implant retained thereto may be performed without the optics and then the optics may be inserted while the implant is still retained withing the sheath. The delivery sheath which may be inserted into the patient's urethra may enclose both the hollow tubes and the endoscopic optical device. The procedure may be observed via the optical device. In some embodiments, this has the advantage that the sheath may need to be inserted only once, since the entire procedure is simultaneously accompanied by visual observation via the endoscopic optic assembly. Additionally, the implant may be placed directly in the desired location. For example, this may obviate the need to insert it blindly into the bladder and then to retract it into position.

In some embodiments, any or all of the sheath and/or the retainer tube and/or the retainer sleeve and/or the optical assembly may be rigid and/or have a controlled bendability and/or longitudinal rigidity and/or spiral flexibility and/or single axis flexibility.

In some embodiments, the sheath and/or optics of a system of the current invention may include various kinds of Urethral endoscopes. For example, they may include some or all of the following general characteristics:
Length:
An insertion sheath and/or a tube of an optical assembly may range from approximately 12 cm to 35 cm or more, depending on the design and application.
Diameter:
An insertion sheath and/or a tube of an optical assembly may range in diameter between 2 mm 5 mm and/or between 5 mm to 15 mm.
An insertion sheath and/or a tube of an optical assembly may be Semi-rigid or flexible e.g., to bend easily, offering better maneuverability during procedures. Alternatively or additionally, an insertion sheath and/or a tube of an optical assembly may be rigid.
An insertion sheath and/or a tube of an optical assembly may be equipped with optical systems for visualization. They may use a straight scope and/or optical fibers and/or newer imaging technologies like charge-coupled device (CCD) or CMOS cameras.
Sheath Material:
The sheath of the urethral endoscope is typically made from materials like stainless steel, plastic, or a combination of materials.

In some embodiments a lumen and/or working channel of a delivery sheath in accordance with the current invention may range between 2 to 3 mm and/or between 3 to 6 mm and/or between 3 to 5 mm. In some embodiment the sheath may have only one lumen, alternatively or additionally the sheath may have two, three or more lumens (e.g., working channels). Optionally, a retainer for a stent and/or an optical assembly may pass into the urethra through a single lumen.

### Exemplary Embodiments

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Some embodiments of the invention have some or all of the following advantages:
1) The implant may be inserted under visual observation.
2) The implant may be placed directly in the desired location in an antegrade fashion. For example, without insertion of the implant into the bladder and/or drawing the implant back from the bladder into the desired location.
3) The entire procedure (in some embodiments after measuring which may include a further preliminary endoscope insertion) may be performed with only one endoscope insertion/removal.
4) The endoscope catheter (which may also be called a delivery sheath) and optics may be retracted while holding the implant in place under vision until the implant is left in place.
5) Damage to patient tissue may be reduced.
6) Patient recovery time may be reduced.
7) Patient complications may be reduced.
8) Implant may be held by the tubes and/or loops of the device with improved stability.
9) Multiple points on the implant may be held. This may facilitate improved control of positioning the implant inside the patient (for example rotation around a longitudinal axis) and/or return of the implant to the catheter.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

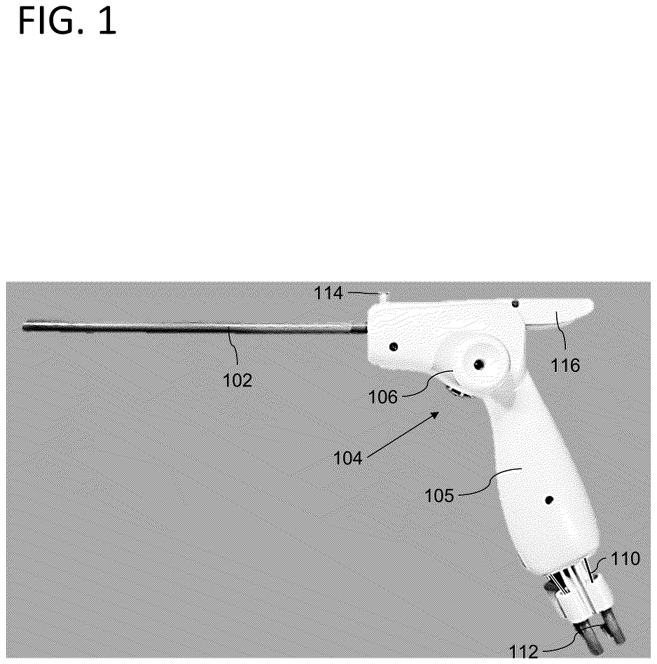

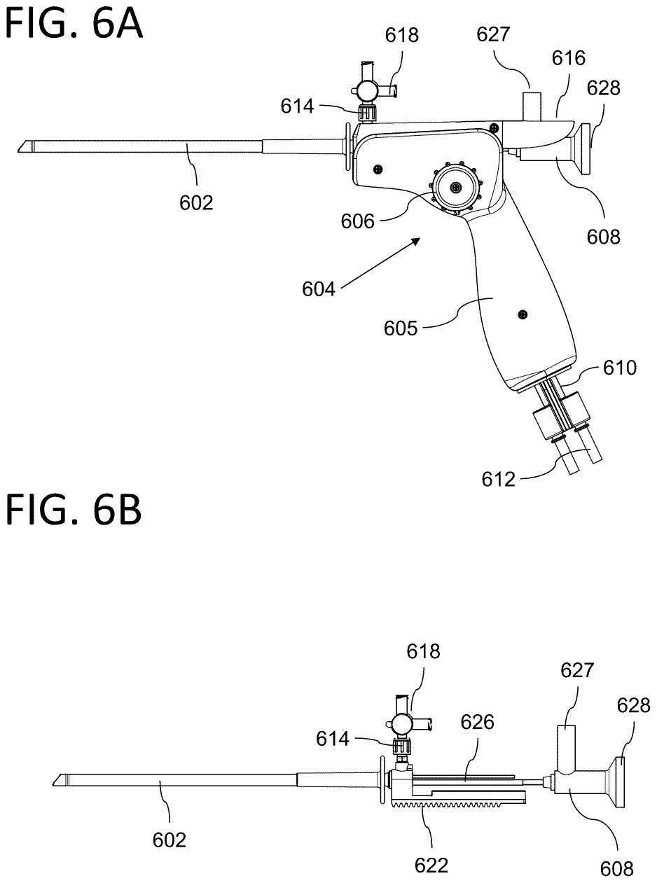

FIG. 1 is an image of a delivery system with a catheter in an extended configuration in accordance with an embodiment of the current invention. In some embodiments, the delivery sheath 102 and/or an implant (e.g., collapsable implant 120) are inserted into the urethra with delivery sheath 102 extended distally. For example, during insertion an implant (e.g., collapsable implant 120) may be contained in a collapsed form in a distal portion of delivery sheath 102. Optionally, the handle 104 includes an extension control (for example a control wheel 106) to extend the delivery sheath 102 distally and/or retract the delivery sheath 102 proximally. Optionally, the delivery system includes an irrigation port 114 and/or a guide 116 for an optical system (e.g., optical assembly 608 as illustrated in later figures (e.g., FIG. 6B)).

In some embodiments, the handle 104 may include a release mechanism for the implant. For example, the implant may be held to the delivery system by a thread 110. For example, the thread 110 may be looped around a portion of the implant at a distal portion of the delivery sheath 102 and/or the free ends of the thread may be held on the controller handle 104, e.g., by one or more anchor 112. Optionally, a release mechanism includes a mechanism to cut the thread 110 and/or release the thread 110 from the anchor 112. For example, a portion of the thread 110 may be exposed which may be cut to release the catheter.

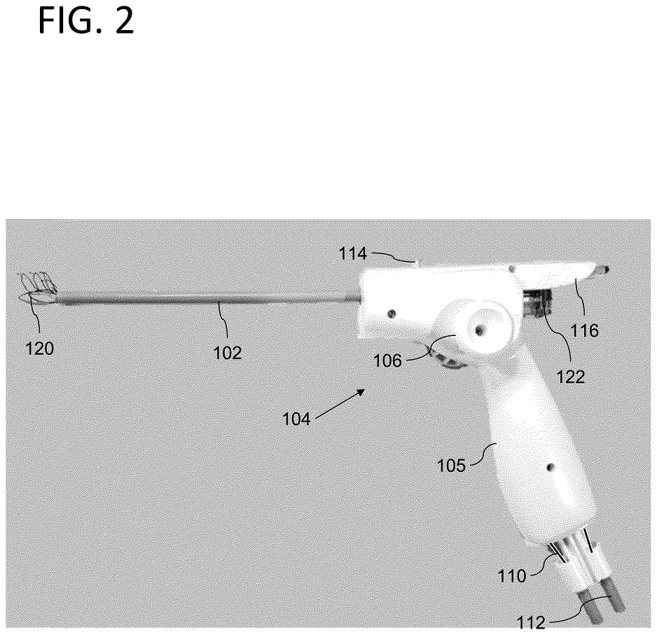

FIG. 2 is an image of a delivery system with a catheter in a partially extended configuration in accordance with an embodiment of the current invention. In some embodiments, in the partially extended configuration, the delivery sheath 102 has been retracted proximally toward handle 104. Optionally, retracting delivery sheath 102 deploys a collapsable implant 120. For example, when delivery sheath 102 is in the extended state, the collapsable implant 120 is secured in a collapsed configuration in a distal portion of the delivery sheath 102. Optionally, while the delivery sheath 102 is retracted, collapsable implant 120 is secured in the same position and/or exposed out the distal end of the delivery sheath 102. Once collapsable implant 120 is released from the delivery sheath 102, collapsable implant 120 expands to an expanded configuration. Optionally, in the partially retracted configuration illustrated in FIG. 2, the collapsable implant 120 is partially collapsed.

In some embodiments, delivery sheath 102 is retracted proximally by rotating control wheel 106 in a first direction and/or extended distally by turning the control wheel 106 in a second direction opposite the first direction. Alternatively or additionally, there may be a different control for retracting and/or extending delivery sheath 102. For example, a control lever (e.g., a trigger) may be connected to the grip 105 of handle 104 to facilitate one handed retraction of the delivery sheath 102 and/or another lever (e.g., control wheel 106) used for extending delivery sheath 102.

In some embodiments, retracting the delivery sheath 102 also retracts the irrigation port 114 and a trolley 122. For example, the irrigation port 114 and trolley 122 are rigidly connected to the delivery sheath 102. In the exemplary embodiment of FIG. 2, irrigation port 114 is shown having retracted proximally with respect to its position illustrated in FIG. 1. In the exemplary embodiment of FIG. 2, trolley 122 is shown having retracted proximally out from the proximal end of handle 104.

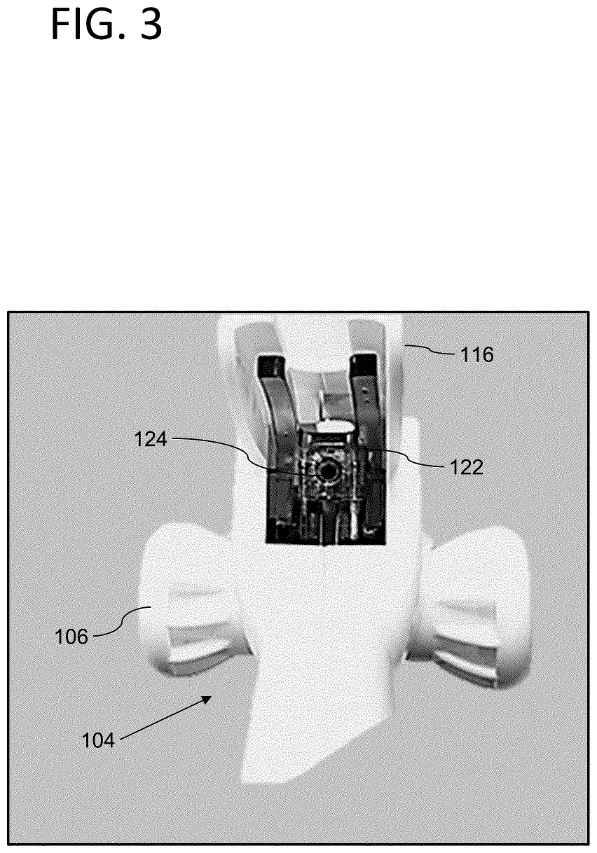

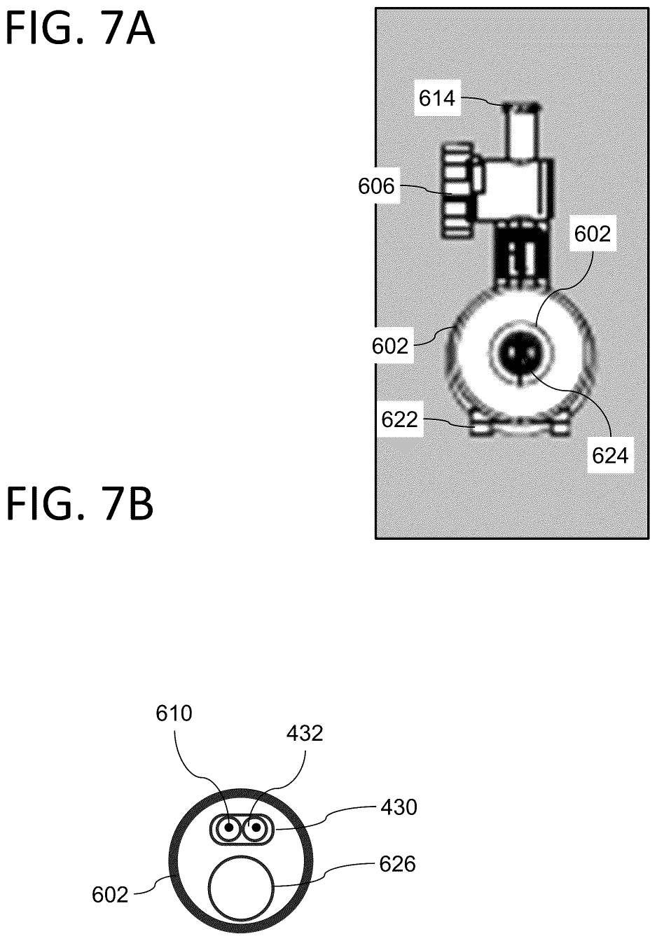

FIG. 3 is an image of a proximal side of a delivery system in accordance with an embodiment of the current invention. In some embodiments, the delivery sheath 102 includes a lumen 124. For example, lumen 124 may have a proximal opening. For example, lumen 124 allows insertion of items through a delivery sheath 102 into the urethra. For example, an optical assembly (e.g., optical assembly 608 for example as illustrated in FIGS. 6 and 7).

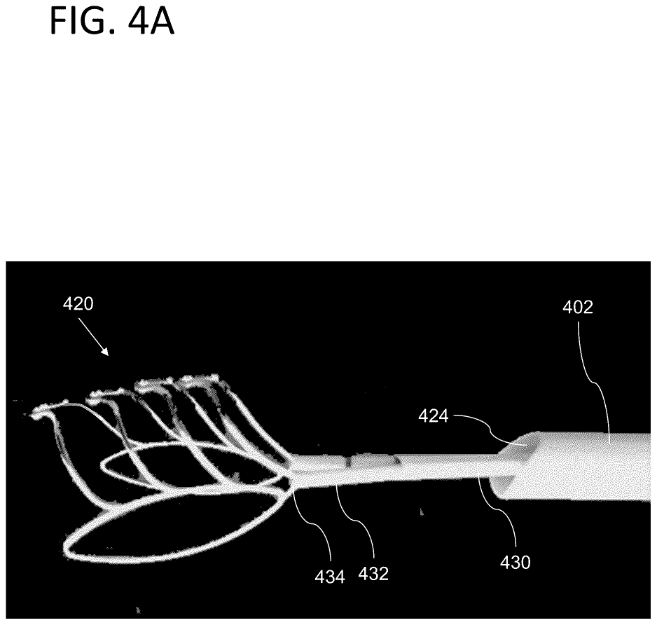

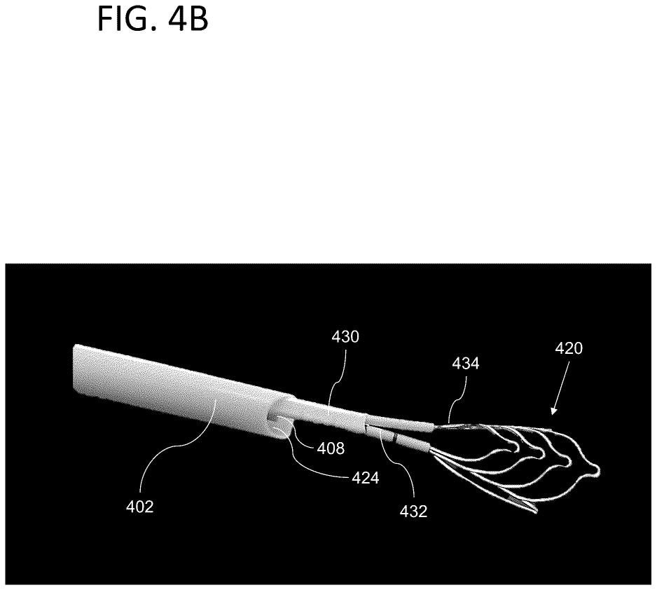

FIG. 4A is an image of a distal end of delivery sheath 402 and implant 420 after retraction of the delivery sheath 402 in accordance with an embodiment of the current invention. In some the implant 420 is retained in place by one or more retainer sleeves 432. For example, one or more proximal corners 434 (e.g., a pair of proximal corners 434) of the implant 420 may be attached to one or more retainer sleeves 432. For example, the proximal corner 434 may be attached to a distal end of the retainer sleeve 432. In some embodiment, attachment is by tying proximal corner 434 to the retainer sleeve 432 by a thread that loops around the proximal corner 434 for example when the thread is kept under tension. Optionally, a proximal portion of the retainer sleeves 432 are held in an outer sleeve 430 that passes through a lumen 424 of the delivery sheath 402. For example, outer sleeve 430 may include a flexible shrink sleeve holding retainer sleeves 432 tightly together and/or giving them a controlled bendability and/or longitudinal rigidity and/or spiral flexibility and/or single axis flexibility facilitating insertion through the delivery sheath 602. Alternatively or additionally, outer sleeve 430 and/or retainer sleeves 432 may be stiff and/or rigid.

FIG. 4B is an image of a distal end of delivery sheath 402 and implant 420 after retraction of the delivery sheath 402 in accordance with an embodiment of the current invention. Optionally, an optical system 408 is inserted into the same lumen as the implant 420. In FIG. 4B a distal portion of an optical system 408 is visible in the distal portion of the delivery sheath 102. For example, the optical system 408 may be used to view deployment of the implant 420 in real time.

In some embodiments, the delivery system may include a control handle (e.g., similar to handle 104) and/or a control wheel (e.g., similar to control wheel 106). For example, the control wheel may be used to control the extension and retraction of the delivery sheath 402 and/or the optical system 408. Additionally or alternatively, the system may be fitted with a trigger control for retraction of the delivery sheath 402. For example, the trigger may be used for one handed retraction of the delivery sheath 402 and/or the optical system 408 and/or exposure of the implant 420. Alternatively or additionally, in after extension of the delivery sheath 402 by means of a trigger, the control wheel may be used to re-extend the delivery sheath 402 and/or to return the implant 420 to the delivery sheath 402 (for example, if the operator decides to reposition the implant).

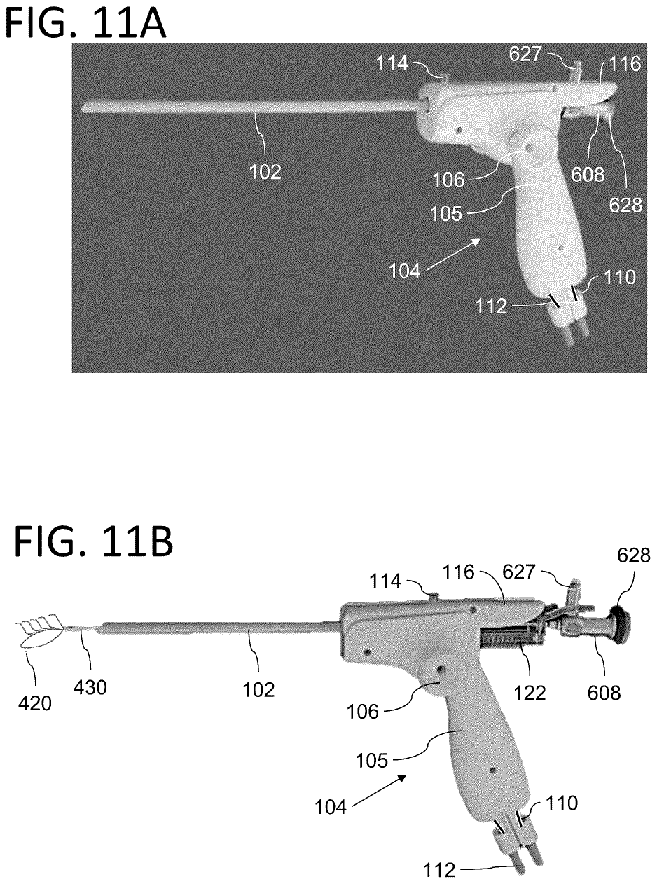

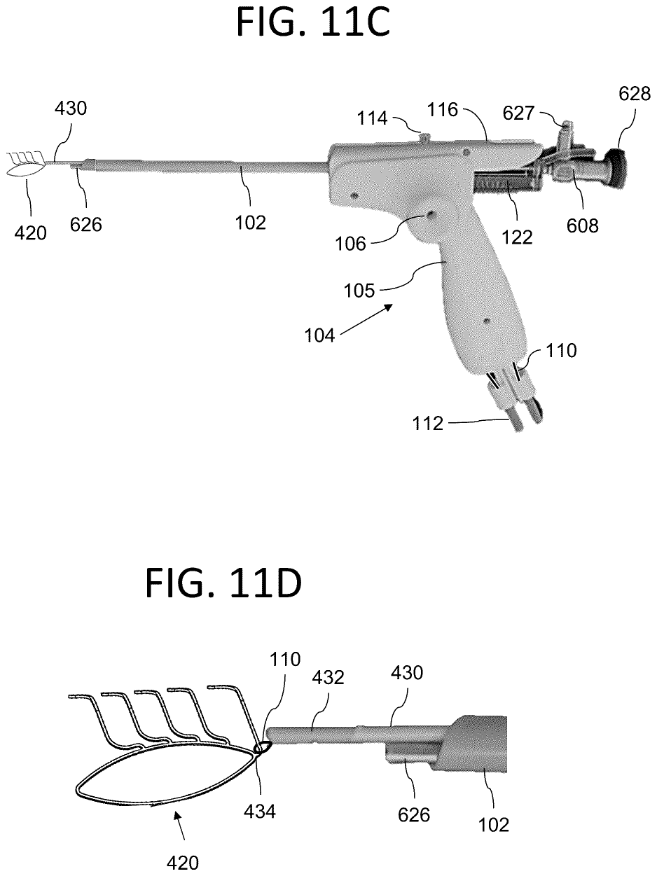

In some embodiments, the optical system 408 may have an additional degree of freedom. For example, the optical system 408 may be repositionable in delivery sheath 402. Optionally, the default position of optical system 408 and delivery sheath 402 is as shown in FIG. 4B wherein the distal end of optical system 408 is proximal to the distal opening of the lumen 424 of delivery sheath 402. For example, with optical system 408 in the default position there is space in the distal portion of lumen 424 to store the implant 420 in a collapsed state (e.g., while inserting delivery sheath 402 into the urethra). Optionally, optical system 408 may be pushed further into delivery sheath 402 to a more advanced position. For example, the advanced potion, the distal end of the optical system 408 may protrude out the distal opening of lumen 424 (e.g., as illustrated in FIGS. 11C and 11D. For example, this may be useful for viewing the implant 420 close up after it has been deployed and/or before it has been released from retainer sleeves 432 e.g., to check that implant 420 is positioned properly. In some embodiments, after positioning of implant 420, while the implant 420 is still connected to retainer sleeves 432, the user may re-extend delivery sheath 402. thereby re-collapsing the implant 420 and/or re-inserting the implant 420 into delivery sheath 402. For example, this may facilitate removal of implant 420 and/or repositioning of implant 420.

In some embodiments the implant may be affixed to one or more retainers (e.g., proximal corners 434 of implant 420 connected retainer sleeves 432) and/or retained at the distal end of the device (e.g., a distal portion of lumen 424). The retainer may be enclosed and/or inserted into a urethra in a delivery sheath (e.g., lumen 424 of delivery sheath 402). The implant may be retained in the delivery sheath by the retainer, and/or the implant may be inserted into the urethra and/or held in a compressed state in the delivery sheath (e.g., in a distal portion of lumen 424 of delivery sheath 402). Additionally or alternatively, the optics (e.g., optical system 408) may be inserted into a urethra through a lumen of a delivery sheath (e.g., through lumen 424 of delivery sheath 402).

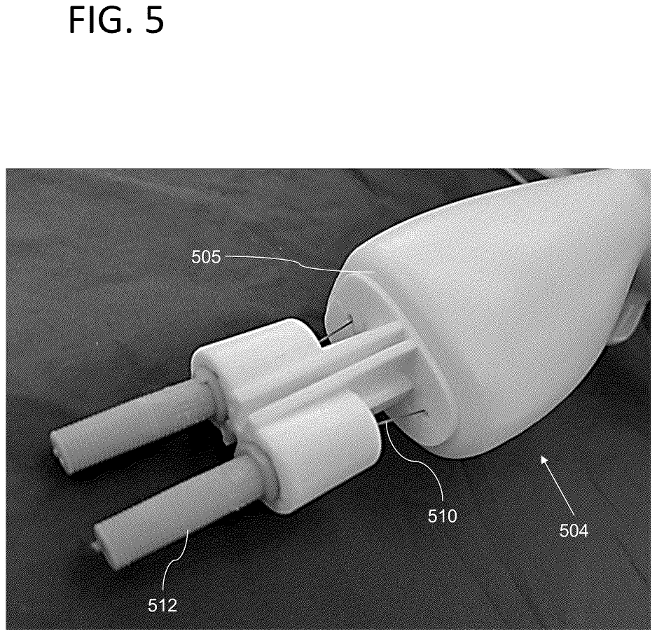

FIG. 5 is an image of a handle of an implant delivery system in accordance with an embodiment of the current invention. In some embodiments, the implant may be stably secured to the retainer via thread 510. For example, the retainer may include a tube (e.g., retainer sleeves 432) and/or thread 510 may that are threaded through the tube and/or looped around the implant (e.g., proximal corners 434 thereof for example as illustrated in FIGS. 4A, 4B, 11C, 11D and/or 12A). Optionally, the free ends of the thread 510 are tensioned to and/or held to the grip 505 of handle 504. Optionally, thread 510 holds the implant in place against a distal end of the retainer. In some embodiments, thread 510 may include standard surgical suture threads made of, for example, synthetic polymer fibers.

In some embodiments, a first end of the thread 510 may be threaded through the retainer (e.g., retainer sleeve 432) from the proximal end to the distal end. Then the first end of the thread is optionally, looped around the implant (e.g., proximal corners 434 of implant 420). Optionally, the first end of the thread 510 then may be passed back through the retainer to handle 504. In some embodiments, the thread 510 is tensioned to secure the implant to the retainer. Alternatively or additionally, the loop may be passed around the implant and then both free ends passed through the retainer sleeve 432. Optionally, a free end of the thread 510 is held by an anchor 512. Optionally, once the implant is positioned in the Urethra, the implant may be released by cutting one leg of the thread 510 at proximal portion of the device. The thread 510 may be cut manually (for example: a protruding portion of the thread 510 with a scissor, etc.) and/or the thread 510 may be cut with a cutter (e.g., built into the handle 504 of the system). Once the thread 510 is cut, it may be removed, for example, by pulling it out of the retainer through the handle 504.

FIG. 6A is schematic illustration of a delivery system with the sheath in an extended configuration in accordance with an embodiment of the current invention. In some embodiments, the delivery sheath 602 and/or a collapsable implant are inserted into the urethra with delivery sheath 602 extended distally. For example, during insertion a collapsable implant may be contained in a collapsed form in a distal portion of delivery sheath 602. Optionally, the handle 604 includes a control wheel 606 to extend the delivery sheath 602 distally and/or retract the delivery sheath 602 proximally. Optionally, the delivery system includes an irrigation port 614 and/or a guide 616 for a trolley 622.

In some embodiments, the handle 604 may include a release mechanism for an implant. For example, the implant may be held to the delivery system by a thread 610. For example, the thread 610 may be looped around a portion of the implant at a distal portion of the delivery sheath 602 and/or the free ends of the thread may be held on the controller handle 604, e.g., by one or more anchor 612. Optionally, a release mechanism includes a mechanism to cut the thread 610 and/or release the thread 610 from the anchor 612. For example, a portion of the thread 610 may be exposed which may be cut to release the catheter.

In some embodiments, after insertion into the urethra, the delivery sheath 602 may been retracted proximally toward handle 604 to a partially retracted position. Optionally, retracting delivery sheath 602 deploys a collapsable implant. For example, when delivery sheath 602 is in the extended state, the collapsable implant is secured in a collapsed configuration in a distal portion of the delivery sheath 602. Optionally, while the delivery sheath 602 is retracted, collapsable implant is secured in the same position and/or exposed out the distal end of the delivery sheath 602. Once collapsable implant is released from the delivery sheath 602, collapsable implant expands to an expanded configuration.

In some embodiments, delivery sheath 602 is retracted proximally by rotating control wheel 606 in a first direction and/or extended distally by turning the control wheel 606 in a second direction opposite the first direction. Alternatively or additionally, there may be a different control for retracting and/or extending delivery sheath 602. For example, there may be a control level (e.g., a trigger) facilitated one handed retraction of the delivery sheath 602 and/or another lever (e.g., control wheel 606) used for extending delivery sheath 602.

In some embodiments, retracting the delivery sheath 602 also retracts the irrigation port 614 and a trolley 622. For example, the irrigation port 614 and trolley 622 are rigidly connected to the delivery sheath 602.

FIG. 6B is schematic illustrations of a trolley and optical assembly in accordance with an embodiment of the current invention. In some embodiments, the trolly includes a transmission (e.g., a pinion rack that engages a gear of control wheel 606). Trolley 622 is used to move delivery sheath 602. For example, trolley 622 is rigidly connected to delivery sheath 602 and/or delivery sheath 602 is supported by trolley 622. Optionally, optical assembly is slidably engaged to sheath delivery sheath 602 and/or trolley 622. For example, the rod 626 of optical assembly 608 may pass through delivery sheath 602. Optionally, optical assembly 608 can engage trolley 622 in multiple positions. For example, a proximal portion of optical assembly 608 may be held by trolley 622 in a forward position in which the distal end of optical assembly 608 (e.g., the distal end of rod 626) protrudes out of a distal opening in delivery sheath 602. For example, the proximal portion of optical assembly 608 may be held by the trolley 622 in a rearward position in which the distal end of the optical assembly 608 (e.g., the distal end of rod 626) is within the lumen of sheath 602. In some embodiments, the proximal portion of optical assembly 608 may be held in a middle position in which the distal end of optical assembly 608 is flush with the distal end of delivery sheath 602. In some embodiments, optical assembly 608 is moved with respect to trolley 622 manually (e.g., the user holds and/or pushes optical assembly 608 into and/or out of delivery sheath 602 with his hand). Alternatively or additionally, there may be an actuator that moves optical assembly 608 with respect to trolley 622 and/or delivery sheath 602. In some embodiments, optical assembly 608 is held to trolley 622 and/or delivery sheath 602(for example, by simple friction and/or pressure) such that optical assembly 608 moves with trolley 622 and/or delivery sheath 602. Alternatively or additionally, there may be any sort of actuator for example, a linear motor and/or a magnetic actuator and/or a slider button and/or a spring driven assembly. For example, a button may release a latch and/or allow a spring to push delivery sheath 602 backward in relation to trolley 622 and/or a linear actuator (e.g., the rack and pinion system of control wheel 606 and trolley 622). Optionally the position of delivery sheath 602 and/or trolley 622 may be locked with respect to a grip 605 of controller handle 604. Optionally the position of optical assembly 608 may be locked with respect to delivery sheath 602 and/or trolley 622 and/or grip 605.

In some embodiments optical assembly 608 is a convention urethral telescope including for example a light post 627 and/or lenses and/or a hollow rod 626 and/or an eyepiece 628. Alternatively or additionally, the system may include a different kind of optical system, for example, including a camera. In some embodiments, trolley 622 includes an irrigation port 614 in communication with the lumen of delivery sheath 602. For example, irrigation port 614 may include a connector for example a tubing connector and/or a connector for a standard stopcock 618. Optionally, irrigation port 614 may include a valve or cover to close the opening of irrigation port 614 when desired.

FIGS. 7A and 7B are distant and close-up cross-sectional views of a delivery sheath in accordance with an embodiment of the current invention. In some embodiments, outer sleeve 430 and/or rod 626 pass through a lumen 624 of delivery sheath 602. For example, lumen 624 may be wide enough for the rod 626 and outer sleeve 430 to slide along independently and/or pass by each other. Optionally the trolley 622 and/or delivery sheath 602 includes a stabilizer for rod 626 and/or outer sleeve 430. For example, trolley 622 may include a guide tube which fits snugly to rod 626 stabilizing rod 626 while allowing it to slide in the longitudinal direction. For example, the trolley 622 may be connected to and/or hold a part of optical assembly 608. For example, a distal portion of delivery sheath 602 may include a stabilizer (e.g., a guide hole and/or guide rod) the stabilize movement of outer sleeve 430 and/or a catheter (e.g., so that they slide smoothly longitudinally delivery sheath 602 and/or the distal opening of delivery sheath 602 without deflecting horizontally).

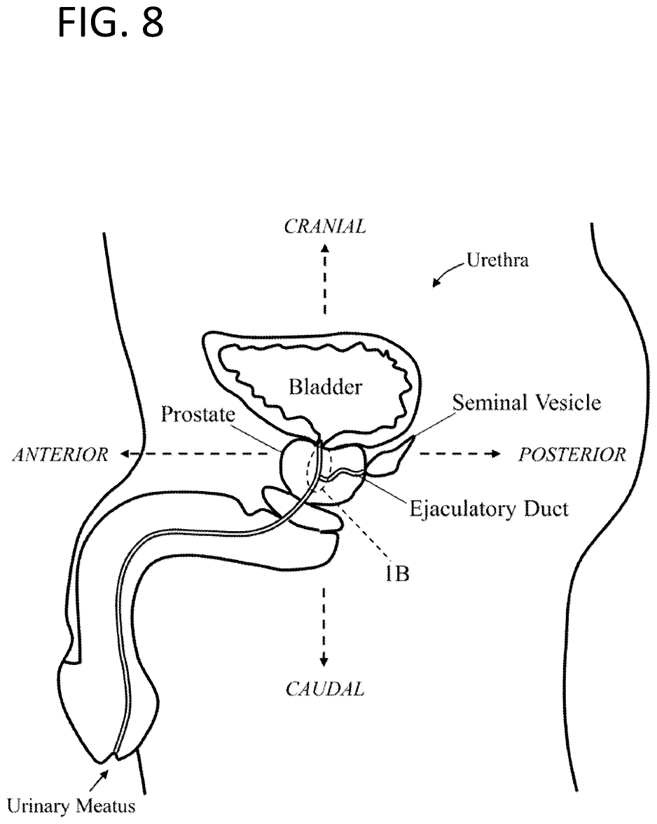

FIG. 8 is a schematic diagram of some relevant areas of the urinary system which are relevant to the medical procedures that are relevant to the current invention. The figure schematically illustrates cross sectional views of a male urethra. The urethra connects the urinary bladder to the urinary meatus for the removal of urine and semen from the body. Furthermore, the prostatic urethra merges about mid length thereof with an ejaculatory duct for carrying semen therein from the seminal vesicle. The prostatic urethra begins at the bladder neck and runs through the prostate approximately 3 cm in length. It begins almost vertically (in a caudal direction) from the bladder then curved anteriorly.

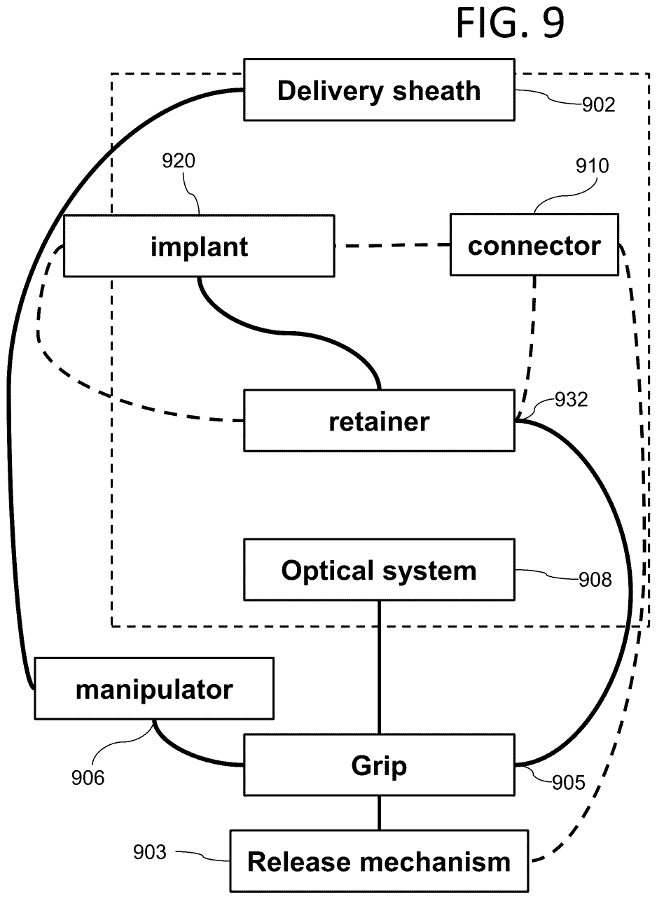

FIG. 9 is a block diagram of an inserter system in accordance with an embodiment of the current invention.

In some embodiments, the system has multiple configurations. For example, in an extended configuration a delivery sheath 902 may be extended distally away from a grip 105 of the handle. For example, in a retracted configuration the delivery sheath 902 may be retracted proximally towards the grip 905 of the handle. In some embodiments, in the extended configuration an implant 920 is held inside delivery sheath 902 for insertion into the urethra. For example, during insertion an implant 920 (e.g., a collapsable implant and/or a non-collapsable implant) may be contained in the delivery sheath 902. Optionally, the handle 104 includes a manipulator 906 to control extension of the delivery sheath 902. For example, the manipulator 906 may include a manual lever and/or control wheel and/or a button to control an automatic actuator and/or a trigger.

In some embodiments, the implant 920 is manipulated by a retainer 932. For example, the retainer 932 may include the connector 910. Optionally, the handle of the system (e.g., grip 905) may include a release mechanism 903 for the implant. For example, the implant may be held to the delivery system by a connector 910. Optionally, a release mechanism 903 includes a mechanism to release the connector 910.

In some embodiments, in a retracted configuration, the delivery sheath 902 is retracted proximally toward handle grip 905. Optionally, retracting delivery sheath 902 exposes the implant 920. For example, when delivery sheath 902 is in the extended state, the implant 920 is secured in a distal portion of the delivery sheath 902. Optionally, while the delivery sheath 902 is retracted, the implant 920 is secured in the same position and/or exposed out the distal end of the delivery sheath 902.

In some the implant 920 is manipulated by one or more retainers 932. For example, the implant 920 (e.g., one or more proximal corner thereof) may be attached to the retainer 932 by the connector 910. For example, the retainer may include a sleeve through which passes the connector (e.g., a thread) and the implant 920 may be tied to the retainer 932 by the thread. In some embodiment, attachment is by tying the implant 920 to the retainer sleeve 432 by a thread that loops around the implant and/or is kept under tension. Optionally, the retainer 932 passes through a lumen of the delivery sheath 902. For example, the retainer 932 may have a controlled bendability and/or longitudinal rigidity and/or spiral flexibility and/or single axis flexibility facilitating insertion through the delivery sheath 602. Alternatively or additionally, retainer 932 may be stiff and/or rigid. Optionally, an optical system 908 is inserted into the urethra through same lumen of the delivery sheath as the implant 920. For example, the optical system 908 may be used to view deployment of the implant 920 in real time.

In some embodiments, the delivery system may include manipulator 906 (e.g., a control wheel and/or a trigger and/or a switch). For example, the manipulator 906 may be used to control extension and/or retraction of the delivery sheath 002 and/or the optical system 908. For example, the system may be fitted with a trigger releasing a spring mechanism for retraction of the delivery sheath 902. Alternatively or additionally, the system may include a switch controlling a motor and/or linear actuator that drives retraction and/or extension of the delivery sheath 902. For example, the trigger and/or switch may be used for one handed retraction of the delivery sheath 902 and/or the optical system 908 and/or exposure of the implant 920. Alternatively or additionally, in after extension of the delivery sheath 902 by means of a trigger and/or switch while a control wheel may be used to re-extend the delivery sheath 902 and/or to return the implant 920 to the delivery sheath 902 (for example, if the operator decides to reposition the implant).

In some embodiments, the optical system 908 may have an additional degree of freedom. For example, the optical system 408 may be repositionable in a lumen of delivery sheath 902. Optionally, in the default position of optical system 908 the distal end of optical system 908 is proximal to the distal opening of the lumen of delivery sheath 902. For example, with optical system 908 in the default position there is space in the distal portion of lumen to store the implant 920. Optionally, the implant 920 is collapsible and/or the space is for storing the implant 920 in a collapsed state. For example, the implant 920 is stored in the lumen while inserting delivery sheath 902 into the urethra. Optionally, optical assembly 608 may be pushed further into delivery sheath 602 to a more advanced position. For example, the advanced potion, the distal end of the optical assembly 608 may protrude out the distal opening of lumen of the sheath 902. For example, this may be useful for viewing the implant 920 close up after it has been deployed and/or before it has been released from retainer 932 e.g., to check that implant 920 is positioned properly. In some embodiments, after positioning of implant 920, while the implant 920 is still connected to retainer 932, the user may re-extend sheath 902 thereby re-inserting the implant 920 into delivery sheath 902. For example, this may facilitate removal of implant 920 and/or repositioning of implant 920.

In some embodiments, the optics (e.g., optical system 908) may be inserted into a urethra through a lumen of a delivery sheath 902.

In some embodiments, the implant may be stably secured to the retainer 932 via connector 910. For example, the retainer 932 may include a tube and/or thread (e.g., connector 910) may that are threaded through the tube and/or looped around the implant (e.g., proximal corners thereof). Optionally, the free ends of the thread (e.g., connector 910) are tensioned to held to the handle grip 905. Optionally, thread 510 holds the implant in place against a distal end of the retainer. In some embodiments, thread 510 may include standard surgical suture threads made of, for example, synthetic polymer fibers.

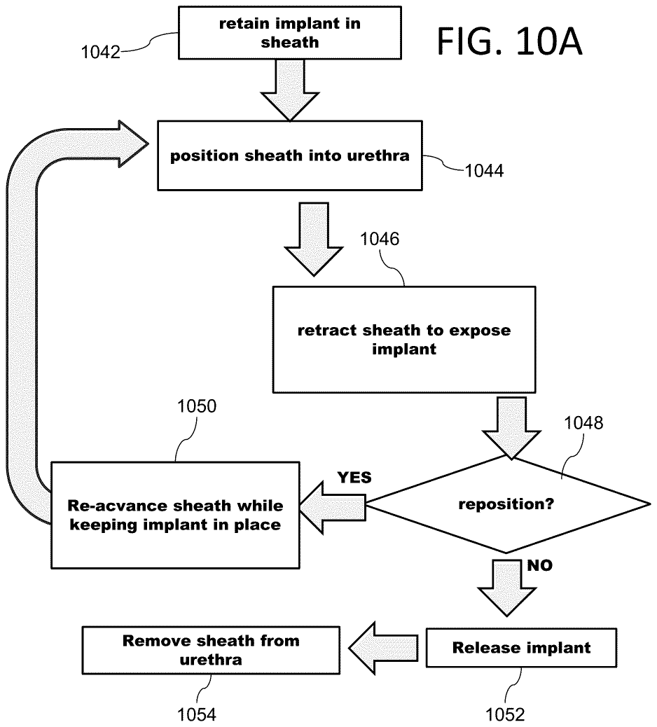

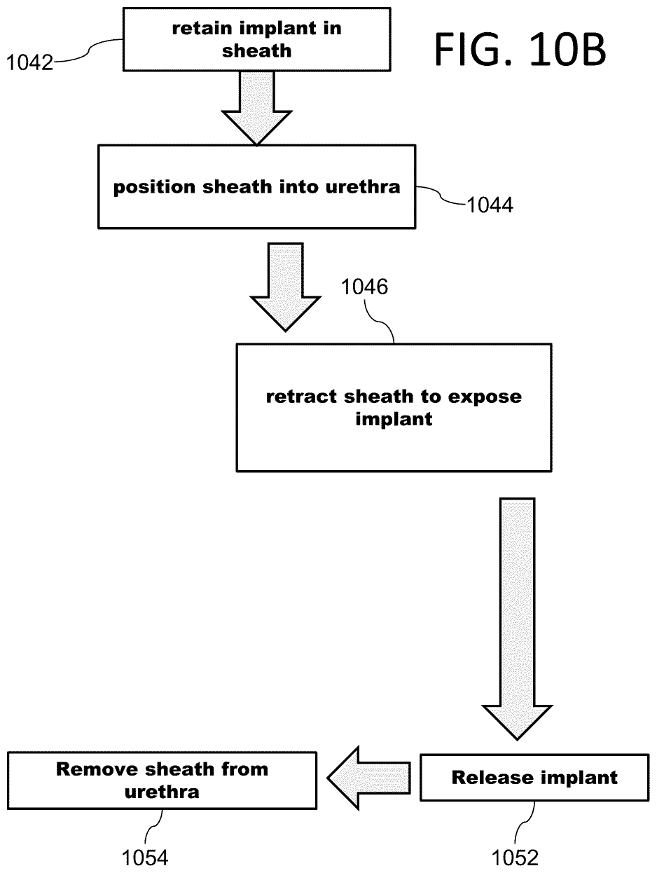

FIGS. 10A and 10B are flow chart illustrations of methods in accordance with embodiments of the current invention. In some embodiments, an implant may be retained 1042 in the distal end of a sheath. Optionally, the sheath and attached implant may be positioned 1044 into the prostatic urethra of the subject. Alternatively or additionally, the implant may be passed through from the proximal side of the sheath to the distal side while the sheath in in the urethra. An optical system may be inserted into the sheath (e.g., through a proximal opening of the sheath) either before or after inserting the sheath into the urethra while the implant remains in the sheath. In some embodiments, the distal end of the optical system may remain proximal to the implant, The sheath may be retracted (e.g., using a control wheel) while the implant is held in place (e.g., by a retainer) thereby exposing 1046 the implant in the urethra. Optionally, the optical system may move along with the sheath. Alternatively or additionally, the optically system may be retained in place as the sheath moves. In some embodiments, all or some of the procedure may be observed visually via the optical system. Optionally, after the

In some embodiments, a user checks the positioning 1044 of the implant. For example, the user may advance the optics to get a closer look at the implant and/or its position in the urethra. If a caretaker chooses 1048 to remove the implant and/or reposition the implant, this may be done by deployment system. For example, the sheath is re-advance 1050 back over the implant while the implant is retained in position (e.g., with the retainer). With the implant back in the sheath, it may be moved to a new position and/or re-exposed. In some embodiments, once the implant has been positioned 1044 and/or exposed 1046 (e.g., the implant is no longer inside the distal end of the sheath), the optical system may be advanced further into the sheath and/or the distal end of the optical system may be advanced out from the distal end of the sheath. This may facilitate closer visual observation of the implant to check that it is properly installed. In some embodiments, the implant may be released 1052 from the endoscope, for example after it has been properly positioned 1044 and/or exposed 1046 the implant may be released 1052. For example, the implant may be released by cutting one end of a loop of thread holding the implant to the retainer and/or pulling the other end of the thread to remove pull the thread out through the catheter. After the procedure, the endoscope may be removed 1054 from the urethra and/or the implant may remain in place.

FIG. 11A-11D illustrate states of a system at various stages in insertion of an implant in accordance with an embodiment of the current invention.

FIG. 11A illustrates a sheath 102 in an extended configuration in accordance with an embodiment of the current invention. In the extended configuration, the sheath 102 optionally envelopes the implant (e.g., implant 420) and/or the implant is held within the catheter. Optionally, with the sheath 102 extended, the sheath is inserted into the urethra. In some embodiments, the optical assembly 608 is coupled to the deployment system (e.g., the rod 626 of the optical assembly 608 is inserted into the sheath 102) during insertion into the urethra, e.g., to guide insertion. Alternatively or additionally, the optical assembly 608 is dis-coupled from the deployment system until the sheath 102 has been at least partially inserted into the urethra.

FIG. 11B illustrates a sheath 102 in a retracted configuration in accordance with an embodiment of the current invention. In some embodiments, while the sheath 102 is inside the urethra, the sheath 102 is retracted while the handle grip 105 is held steady. For example, holding the handle grip 105 steady will hold the implant 420 steady. Pulling back the sheath 102 optionally exposes the implant 420 (e.g., as illustrated in FIG. 11B). In some embodiments, once exposed, implant 420 expands into place in the urethra. Optionally, the sheath 102 is retracted by turning control wheel 106. Alternatively or additionally, the sheath 102 may be retracted with the same hand and that is holding the handle grip 105 (e.g., one handed) for example using a trigger.

FIGS. 11C and 11D illustrate a sheath 102 in a retracted configuration with the optical assembly 608 in an advanced position in a general view and expanded view respectively in accordance with an embodiment of the current invention. In some embodiments, the optical assembly 608 may be pushed further forwards (e.g., as illustrated in FIGS. 11C and 11D). For example, pushing the proximal portion of some embodiment's optical assembly 608 (e.g., light post 627 and/or eyepiece 628) distally into trolley 122 may cause the distal end of the optics (e.g., the distal end of hollow rod 626) to be pushed out of the distal end of the sheath 102. In some embodiments, this may give the operator a better view of the positioning of the implant 420.

In some embodiments, the implant 420 is held at one or more points (e.g., holding two proximal corner 434) to the retainer sleeve 432 by a connector. For example, the connector may include a thread 110 threaded through retainer sleeve 432 and looped around proximal corner 434. Optionally, tensioning thread 110 holds implant 420 to retainer sleeve 432. Optionally holding implant 420 to retainer sleeve 432 facilitates manipulating the implant 420 as necessary to the correct position. For example, holding implant 420 to retainer sleeve 432 may facilitate rotating the implant 420 around its longitudinal axis (e.g., e.g., by rotating outer sleeve 430 around its axis from the proximal end of the controller handle 104, rotating implant 420 may be facilitated by grasping implant 420 on two corners) and/or exposing the implant 420 (e.g., by holding the implant 420 steady while retracting sheath 102) and/or returning the implant 420 to the sheath 102 (e.g., by extending the sheath 102 while holding the implant 420 steady). Optionally at this point (e.g., with the implant 420 remains connected to the retainer sleeve 432) re-extending the sheath 102 reintroduces implant 420 into the sheath 102. Reintroducing implant 420 into sheath 102 may facilitate removing and/or can be repositioning the implant 420.

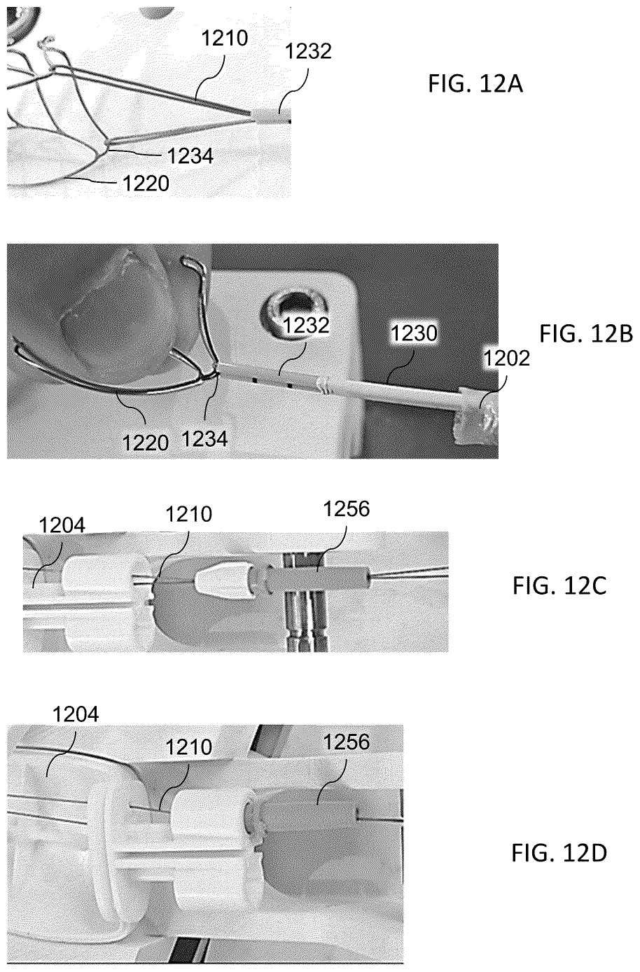

FIGS. 12A to 12D illustrate connecting an implant to a retainer tube with a thread loop in accordance with an embodiment of the current invention.

FIG. 12A illustrates connecting an implant 1220 to a retainer tube 1232 in accordance with an embodiment of the current invention. In some embodiments, retainer tubes 1232 are connected to multiple locations on the implant 1220. Optionally, the connection is made using loops of a thread 1210. For example, the thread 1210 is looped around a portion of the implant 420).

FIG. 12B illustrates tightening an implant 1220 to a retainer tube 1232 in accordance with an embodiment of the current invention. Optionally, free ends of the loops are passed through the distal end of the retainer tube 432. Optionally, the retainer sleeves 432 are held together in an outer sleeve 1230. In some embodiments, tensioning the threads 1210 draws the corners 1234 of the implant 1220 to the retainer tubes and/or draws the corners 1234 together to the outer tube 1230. Optionally this draws the implant 1220 to a collapsed and/or partially collapsed state. In some embodiments, the thread 1210 is passed through a distal opening into the retainer tubes 1232 and/or the outer tube 1230 and/or the sheath 1202.

FIGS. 12C and 12D illustrate anchoring a connector at a handle in accordance with an embodiment of the current invention. In some embodiments, the thread 1210 passes through the retainer tubes 1232 and/or the outer tube 1230 and/or the sheath. For example, the thread 1210 (e.g., the two free ends of the loop) pass out a proximal end of the retainer tubes 1232 and/or of the outer tube 1230 and/or of the sheath 1202 to the handle 1204 of the system. Optionally, at the handle 1204, the thread 1210 is tensioned from the proximal end, pulling the implant 1220 against the distal end of the retainer tubes 1232. One or both of proximal/free ends of the thread 1210 are then, optionally, anchored to the handle 1204 of the system. One of the two free ends is optionally left exposed to cutting (either by an internal cutting apparatus and/or outside the handle 1204 to be cut manually). When the operator is ready to release the implant 1220, he optionally cuts the exposed end of the thread 1210 and/or pulls out the anchor 1256 away from the handle 1204, pulling the thread out through the tube and/or releasing the implant.

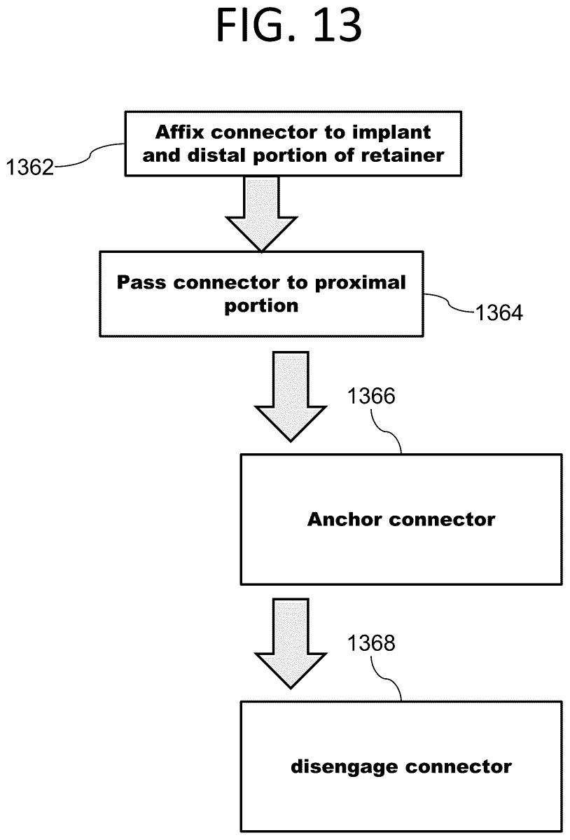

FIG. 13 is a flow chart of a method of affixing an implant to a delivery system in accordance with an embodiment of the current invention. In some embodiments, the implant is affixed 1362 to a distal portion of a retainer at a distal portion of a sheath. For example, the retainer may have a controlled bendability and/or longitudinal rigidity and/or spiral flexibility and/or single axis flexibility facilitating insertion through the delivery sheath and/or retention of the implant while allowing snaking along a path into the body. For example, the retainer may include a flexible tube and/or the implant may be connected to the retainer with a thread (e.g., looping the thread around the implant). In some embodiments, the proximal portion of the retainer and/or the connector (e.g., free ends of the thread) may be passed 1364 through the delivery sheath to its proximal end. In some embodiments, the connector is anchored 1366 at the proximal end of the delivery sheath. (e.g., the thread is tensioned and/or anchored to a handle of the delivery system to secure the implant to the retainer). Optionally, once the implant is positioned in the body, the implant may be released by disengaging 1368 the proximal and of the connector. For example, one leg of the thread may be cut at the handle of the device. Optionally, the connector (e.g., the thread) may be cut manually may be removed, for example, by pulling it out of the retainer through the handle disengaging 1368 the connector and/or releasing the implant.

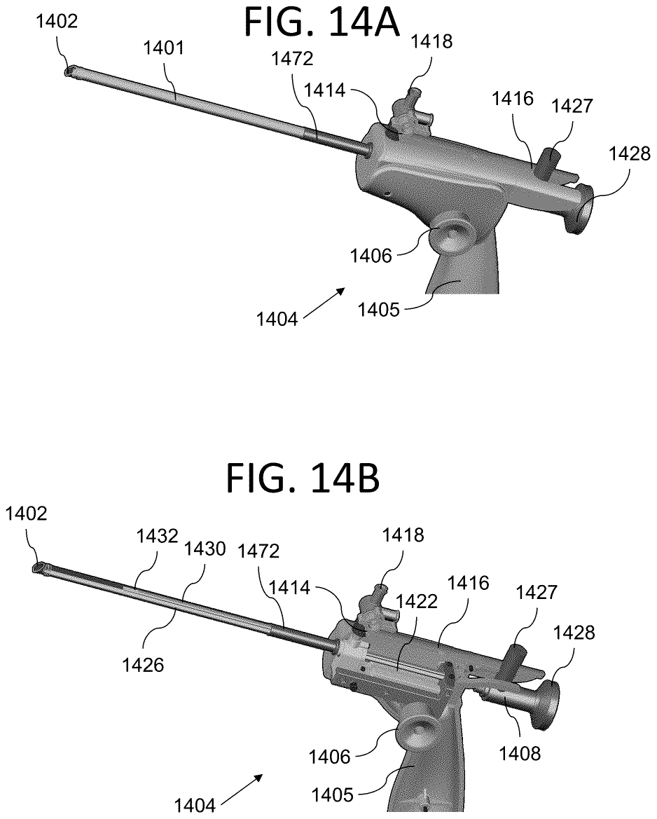

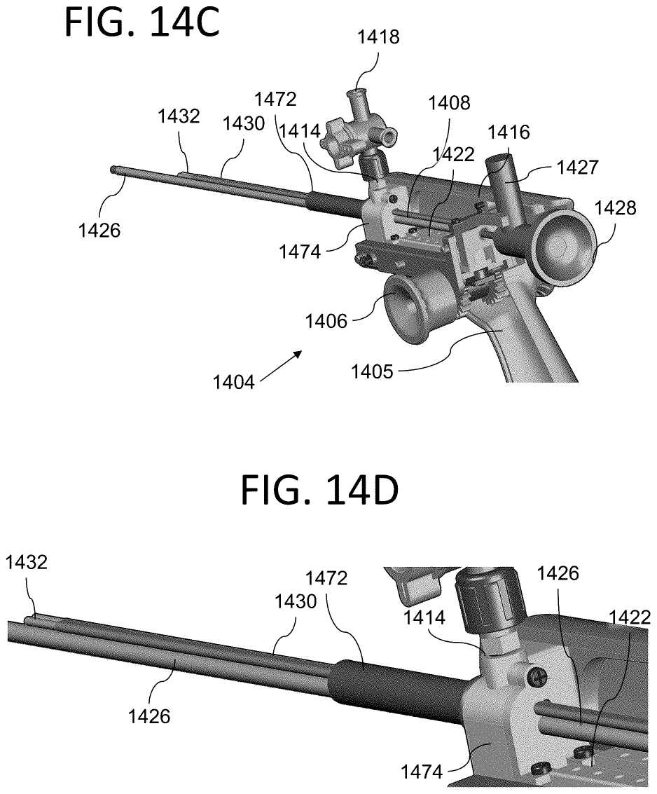

FIG. 14A is schematic illustration of a delivery system with the sheath in an extended configuration in accordance with an embodiment of the current invention. In some embodiments, the delivery sheath 1402 and/or a collapsable implant are inserted into the urethra with delivery sheath 1402 extended distally. For example, during insertion a collapsable implant may be contained in a collapsed form in a distal portion of delivery sheath 1402. Optionally, the handle 1404 includes a control wheel 14014 to extend the delivery sheath 1402 distally and/or retract the delivery sheath 1402 proximally. Optionally, the delivery system includes an irrigation port 1414.

In some embodiments optical assembly 1408 is a convention urethral telescope including for example a light post 1427 and/or lenses and/or a hollow rod (e.g., hollow rod 1426 illustrated in FIG. 14B) and/or an eyepiece 1428. Alternatively or additionally, the system may include a different kind of optical system, for example, including a camera. In some embodiments, trolley 1422 includes an irrigation port 1414 in communication with the lumen of delivery sheath 1402. For example, irrigation port 1414 may include a connector for example a tubing connector and/or a connector for a standard stopcock 1418. Optionally, irrigation port 1414 may include a valve or cover to close the opening of irrigation port 1414 when desired.

In some embodiments, the delivery system includes a guide 1416 for an optical assembly 1408. For example, guide 1416 includes a slot through with passes a light post 1427 of the optically assembly. The guide 1416 is shaped and sized such that when trolley 1422 and sheath 1402 are in the extend position, guide 1416 blocks distal movement of the optical assembly 1408 such that the optical assembly 1408 is blocked from advancing distally. For example, the optically assembly may be locked in a rearward position when the trolley 1422 and/or sheath 1402 are in the extended configuration. For example, this may prevent a user from inadvertently advancing a rod 1426 of the optical assembly 1408 into a distal portion of sheath 1402 and or out of a distal end of the sheath 1402. For example, in some embodiments, advancing rod 1426 while an implant is inside the sheath 1402 may interfere with an implant stored in the distal portion of the sheath 1402 and/or interfere with delivery of the implant.

FIG. 14B is schematic illustration of a delivery system with the sheath shown as transparent in an extended configuration in accordance with an embodiment of the current invention. In the illustration of FIG. 14B, the left-hand external shell of the handle 1404 has been removed to reveal the inner works of the exemplary delivery system. In some embodiments, the trolly includes a transmission (e.g., a pinion rack that engages a gear of control wheel 1406). In some embodiments, a trolley 1422 is used to move delivery sheath 1402. For example, trolley 1422 is rigidly connected to delivery sheath 1402 and/or delivery sheath 1402 is supported by trolley 1422.

Optionally, an optical assembly 1408 is slidably engaged to sheath delivery sheath 1402 and/or trolley 1422. For example, the rod 1426 of optical assembly 1408 may pass through delivery sheath 1402. Optionally, optical assembly 1408 can engage trolley 1422 in multiple positions. For example, in FIGS. 14A-14D the proximal portion of optical assembly 1408 is illustrated in a rearward position in which the distal end of the optical assembly 1408 (e.g., the distal end of rod 1426) is within the lumen of sheath 1402. In some embodiments, optical assembly 1408 is moved with respect to trolley 1422 manually (e.g., the user holds and/or pushes optical assembly 1408 into and/or out of delivery sheath 1402 with his hand). Alternatively or additionally, there may be an actuator that moves optical assembly 1408 with respect to trolley 1422 and/or delivery sheath 1402. In some embodiments, optical assembly 1408 is held to trolley 1422 and/or delivery sheath 1402 (for example, by simple friction and/or pressure) such that optical assembly 1408 moves with trolley 1422 and/or delivery sheath 1402.

In some an implant is retained in place by one or more retainer sleeves 1432. For example, one or more proximal corners of the implant may be attached to one or more retainer sleeves 1432. For example, the proximal corner may be attached to a distal end of the retainer sleeve 1432. In some embodiment, attachment is by tying proximal corner to the retainer sleeve 1432 by a thread that loops around the proximal corner. Optionally, a proximal portion of the retainer sleeves 1432 are held in an outer sleeve 1430 that passes through a lumen of the delivery sheath 1402. For example, outer sleeve 1430 may include a flexible shrink sleeve holding retainer sleeves 1432 tightly together and/or giving them a controlled bendability and/or longitudinal rigidity and/or spiral flexibility and/or single axis flexibility facilitating insertion through the delivery sheath 1402. Alternatively or additionally, outer sleeve 1430 and/or retainer sleeves 1432 may be stiff and/or rigid. In some embodiments, the delivery system may include a secondary sheath 1401. For example, secondary sheath 1401 may be within sheath 1402 and/or surround rod 1426 and/or outer sleeve 1430.

In some embodiments, with sheath 1402 in the extended position, there is space between the distal ends of retainer 1432 and the distal end of sheath 1402. For example, the space may have a length of between 1 mm to 5 mm and/or between 5 mm to 50 mm. For example, an implant may be stored withing the sheath in the space between the distal ends of retainer 1432 and the distal end of sheath 1402.

In some embodiments, there may be any sort of actuator for example, a linear motor and/or a magnetic actuator and/or a slider button and/or a spring driven assembly. For example, a button may release a latch and/or allow a spring to push delivery sheath 1402 backward in relation to trolley 1422 and/or a linear actuator (e.g., the rack and pinion system of control wheel 1406 and trolley 1422). Optionally the position of delivery sheath 1402 and/or trolley 1422 may be locked with respect to a grip 1405 of controller handle 1404. Optionally the position of optical assembly 1408 may be locked with respect to delivery sheath 1402 and/or trolley 1422 and/or grip 1405.

FIGS. 14C and 14D are close-up view of an optical assembly and retainer in a delivery system in an extended configuration in accordance with an embodiment of the current invention. In the illustrations of FIGS. 14C and 14D, the left-hand external shell of the handle 1404 and the sheaths 1401 and 1402 have been removed to reveal the inner works of the exemplary delivery system. In some embodiments, the system may include an insertion guide 1474 and/or a proximal guide tube 1472. For example, insertion guide 1474 and/or proximal guide tube 1472 may hold a proximal portion of rod 1426 and/or sleeve 1430 and/or sheaths 1401 and/or 1402 steady with respect to handle 1404. For example, this may facilitate smooth and/or accurate extending and/or retracting of sheaths 1401 and/or 1402. For example, this may facilitate smooth and/or accurate advancing and/or reward movement of rod 1426 Optionally, guide 1417 may supply a fluid path between port 1414 and a lumen of sheath 1401 and/or 1402.

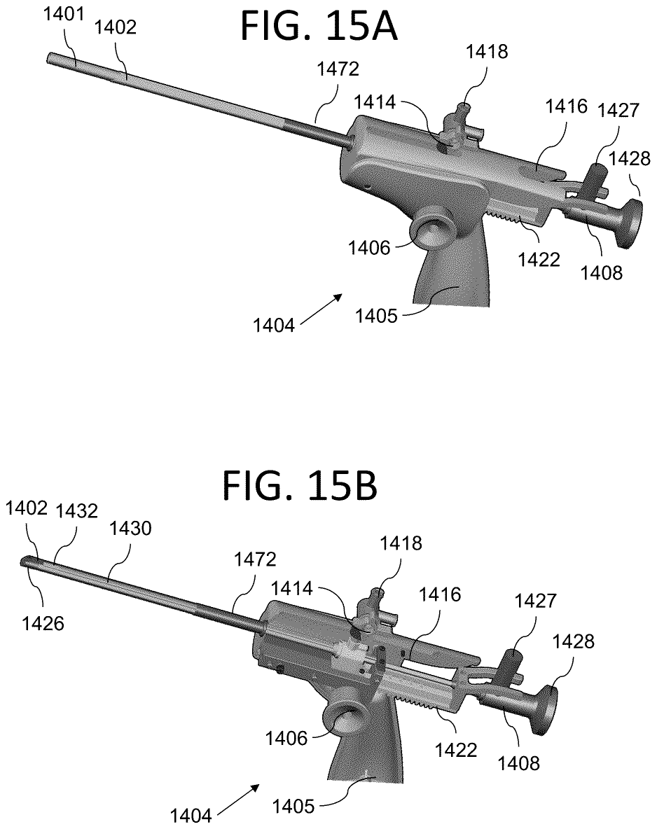

FIG. 15A is schematic illustration of a delivery system with the sheath in a retracted configuration in accordance with an embodiment of the current invention. In some embodiments, after insertion into the urethra, delivery sheath 1402 is retracted. For example, retracting the delivery sheath may deploy an implant into the urethra.

FIG. 15B is schematic illustration of a delivery system with the sheath shown as transparent in the retracted configuration in accordance with an embodiment of the current invention. In the illustration of FIG. 14B, the left-hand external shell of the handle 1404 has been removed to reveal the inner works of the exemplary delivery system.

In some embodiments, after insertion into the urethra, the delivery sheath 1402 may been retracted proximally toward handle 1404 to a partially retracted position. Optionally, retracting delivery sheath 1402 deploys a collapsable implant. For example, when delivery sheath 1402 is in the extended state, the collapsable implant is secured in a collapsed configuration in a distal portion of the delivery sheath 1402. Optionally, while the delivery sheath 1402 is retracted, collapsable implant is secured in the same position and/or exposed out the distal end of the delivery sheath 1402. For example, once collapsable implant is released from the delivery sheath 1402, the collapsable implant may expand to an expanded configuration.

In some embodiments, delivery sheath 1402 is retracted proximally by rotating control wheel 1406 in a first direction and/or extended distally by turning the control wheel 1406 in a second direction opposite the first direction. Alternatively or additionally, there may be a different control for retracting and/or extending delivery sheath 1402. For example, there may be a control level (e.g., a trigger) facilitated one handed retraction of the delivery sheath 1402 and/or another lever (e.g., control wheel 1406) used for extending delivery sheath 1402. Optionally control wheel 1406 may be moved from the left to the right side of the grip 1405 and/or from the left side to the right side (facilitating use by either a right-handed and/or left-handed user). Optionally, the control wheel 1406 may be removed, e.g., to facilitate efficient storage and/or transport of the system.

In some embodiments, retracting the delivery sheath 1402 also retracts the irrigation port 1414 and a trolley 1422. For example, the irrigation port 1414 and trolley 1422 are rigidly connected to the delivery sheath 1402.

In the exemplary embodiment of FIGS. 15A and 15B the optical assembly 1408 is illustrated in a rearward position with respect to trolley 1422. In some embodiments, with the trolley 1422 in the retracted position, guide 1416 does not block advancement of the optical assembly. In some embodiments, when trolley 1422 and/or sheath 1402 are retracted optical assembly 1408 moves backward with the trolley and/or sheath 1402. For example, as sheath 1402 moves, rod 1426 may remain in the same position (e.g., the retracted position) with respect to the sheath 1402. Alternatively or additionally, optical assembly 1408 may not move with sheath 1402 and/or trolley 1422. For example, as sheath 1402 is retracted, rod 1426 may be advanced with respect to the sheath 1402.

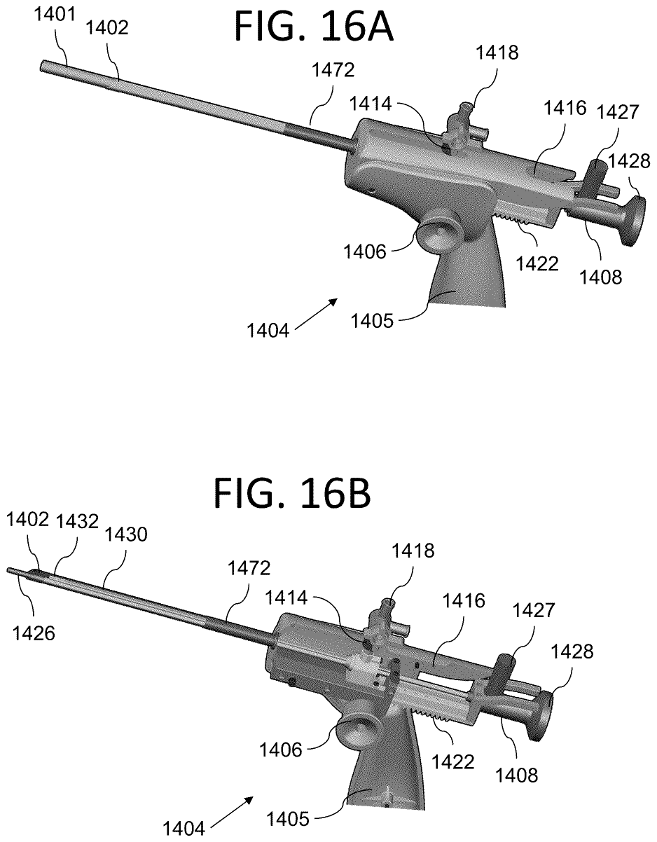

FIG. 16A is schematic illustration of a delivery system with the sheath in a retracted configuration in accordance with an embodiment of the current invention.

FIG. 16B is schematic illustration of a delivery system with the sheath shown as transparent in the retracted configuration in accordance with an embodiment of the current invention. In the illustration of FIG. 14B, the left-hand external shell of the handle 1404 has been removed to reveal the inner works of the exemplary delivery system.

In the exemplary embodiment of FIGS. 16A and 16B the optical assembly 1408 is shown in an advanced configuration. For example, with trolley 1422 and/or sheath 1402 in the retracted position, optical assembly 1408 may be pushed and/or slid distally. For example, this may give a better view of a deployed implant. For example, this may push a distal end of rod 1426 out of a distal opening of sheath 1402.

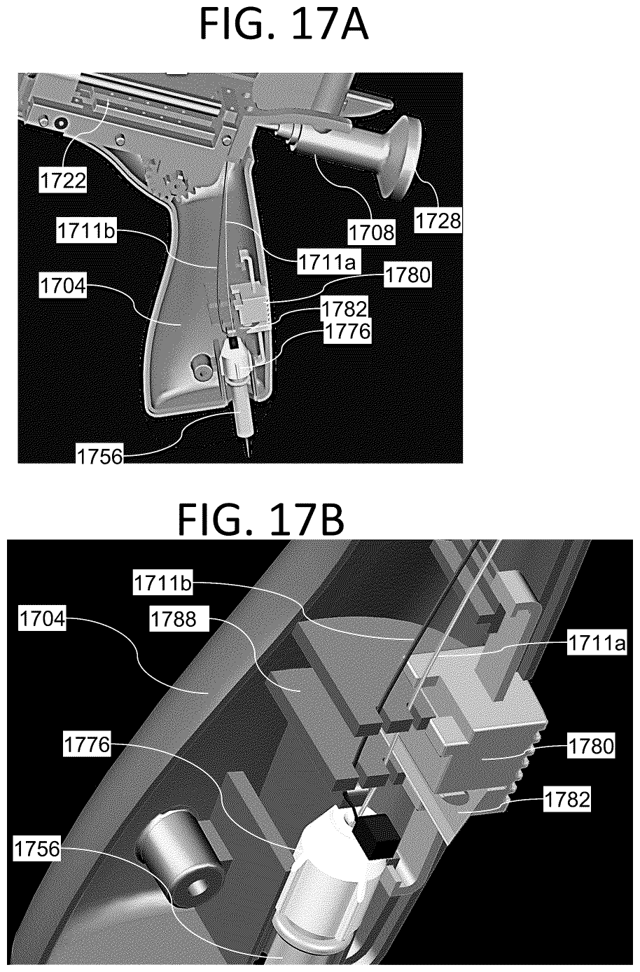

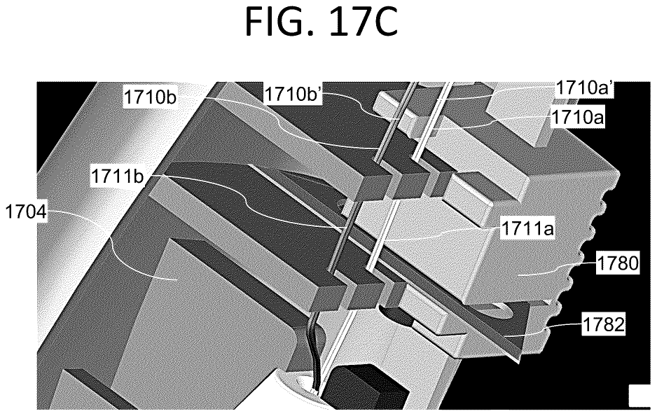

FIGS. 17A-17C are schematic illustrations of a handle of a deployment system including a release module for an implant in accordance with an embodiment of the current invention.

In some embodiments, the implant is affixed to a distal portion of a retainer at a distal portion of a sheath. For example, the retainer may have a controlled bendability and/or longitudinal rigidity and/or spiral flexibility and/or single axis flexibility facilitating insertion through the delivery sheath and/or retention of the implant while allowing snaking along a path into the body. For example, the retainer may include a flexible tube and/or the implant may be connected to the retainer with a thread. For example, one or more loops of the thread may pass around a portion the implant. In some embodiments, the proximal portion of the retainer and/or the connector may be passed through the delivery sheath to its proximal end. For example, one end of each loop of the thread may be gathered into a first collection 1711a of threads and a second end of each loop may be gathered into a second collection 1711b of threads. Optionally, the first and second collections 1711a, 1711b pass through the handle 1704 of the delivery system.

In some embodiments, the connector is anchored at the proximal end of the delivery sheath. (e.g., first collection 1711a and/or second collection 1711b are connected to one or more anchors 1756 and/or to a handle 1704 of the delivery system. For example, the tension on the threads may secure the implant to the retainer.

In some embodiments, once the implant is positioned in the body, the implant may be released by disengaging the proximal and of the connector. For example, one first collection 1711a of threads may be cut. Optionally, the handle includes a cutter assembly. In the exemplary embodiment of FIGS. 17A-17C, the cutter includes a control (e.g., a push button 1780) and/or a cutting tool (e.g., a blade 1782). Optionally, manually pushing the button 1780 pushes blade 1782 to cut thread collection 1711a. Optionally, thread collection 1711a is held in place by a guide 1788 such that it cannot move out of the path of blade 1782. Optionally, the second collection 1711b of threads is positioned such that it is not in the path of blade 1782 and/or will not be cut.

In some embodiments, after one end of the loops is freed (e.g., by cutting collection 1711a), the loops are disengaged from the implant releasing the implant. For example, has been cut and/or disconnected from anchor 1756, the other ends (e.g., collection 1711b) is pulled away from the implant. For example, anchor 1756 may be pulled away from handle 1704. Pulling the second ends of the threads (e.g., collection 1711b) may drawing the free ends (e.g., collection 1711a) of the thread through and/or away from the implant. For example, pulling on collection 1711b after cutting collection 1711b pulls the free (e.g., cut) end of collection 1711a through a sheath and/or through the opening in the implant through which the thread had been looped and/or back out of the sheath. This optionally nullifies the loop and/or releases the implant. Optionally, an adjustment nut 1776 is provided and/or engaged to anchor 1756 to facilitate tensioning the threads.

In FIG. 17A an exemplary optical assembly 1708 including an eyepiece 1728 and rod are shown. An exemplary trolley 1722 is illustrated.

In some embodiments, two loops hold the implant on two locations, each location to one of two retainers. For example, in FIG. 17C, collection 1711a includes two threads 1710a and 1710a′ which are a first leg of a first loop and a second loop respectively. Optionally, the first loop holds a first part of the implant to a first retainer and/or the second loop holds the implant to a second retainer. For example, collection 1711b includes two threads 1710b and 1710b′ which are a second leg of the first loop and the second loop respectively. Alternatively or additionally, a thread anchor may include an elastic tensioning element (e.g., a coil spring may maintain tension on the thread).

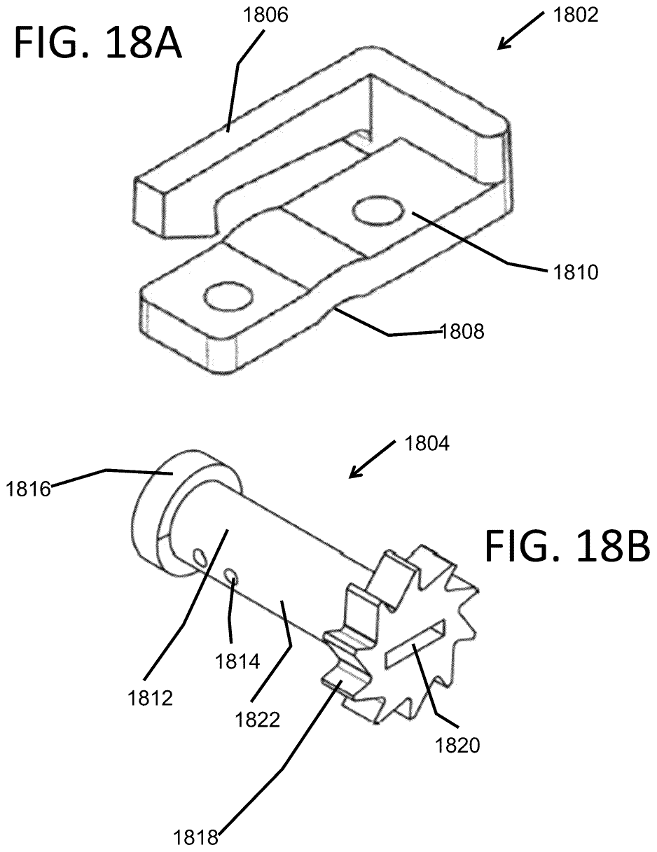

FIGS. 18A and 18B are schematic illustrations of a spool type tensioning element in accordance with an embodiment of the current invention. FIG. 18 illustrates an exemplary tensioning mechanism in accordance with an embodiment of the current invention. In some embodiments, a tensioning system includes a reel 1804 and a bracket 1802. Optionally, the reel 1804 includes multiple sections. For example, the sections may include a spool 1812, an axel 1822 and/or a tension retaining section (e.g., a rack 1818 and/or an elastic tensioner). For example, the spool 1812 is on a distal section of reel 1804. Optionally, the spool 1812 includes flange 1816 preventing the thread from falling off the end thereof and/or a line tie hole 1814 for holding the threads. For example, a middle section of the reel 1804 includes an axel 1822. For example, a proximal section of reel 1804 includes a rack 1818 (ratchet gear). The bracket 1802 optionally includes a U-shaped clamp 1808 with mounting flanges 1810. Optionally a pawl 1806 is formed from one of the mounting flanges 1810. For example, pawl 1806 is an integral portion of the flanges 1810 bent at 90 degrees and shaped to engage the pawl 1806 when the reel 1804 is held in the bracket 1802. Elasticity of the bracket 1802, optionally, facilitates the pawl 1806 to flex into and out of the teeth of the rack 1818 as the reel 1804 rotates. Optionally, the bracket 1802 and/or reel 1804 are each made of a single integral piece of molded plastic. Alternatively or additionally, other materials may be used. For example, the bracket 1802 may be formed of an integral piece of sheet metal and/or various portions may be formed separately and joined together.

In use, the bracket 1802 is oriented perpendicular to the reel 1804. The saddle clamp 1808 optionally surrounds the axel 1822 on three sides. The flanges 1810 are bolted to a surface on opposite sides of the axel 1822 holding the axel 1822 rotatably to the surface. For example, when the bracket 1802 holds the reel 1804 to a surface, the pawl 1806 couples to the rack 1818 to inhibit the spool from rotating backwards.

In some embodiments, reel 1804 includes a slot 1820 on one end for a screw driver to turn the reel 1804 and put tension on the string that is wound around the spool. Alternatively or additionally, an elastic tensioning mechanism (e.g., a torsion spring and/or rubber band) may be used to preserve tension.

For example, each of the parts may be made of a single integral piece of plastic.

It is expected that during the life of a patent maturing from this application many relevant technologies will be developed and the scope of the terms are intended to include all such new technologies a priori.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method of inserting urethral implant storing implant in distal portion of a lumen of a delivery sheath;
inserting the distal portion of the sheath into a urethra;
inserting optics through proximal portion of lumen to view out of a distal opening of lumen while implant remain in distal portion of lumen.

2. The method of claim 1, further comprising retaining the implant on a retainer and retracting the sheath exposing the implant inside the urethra.

3. The method of claim 2, wherein the retainer extends from a proximal opening of the sheath to the implant.

4. The method of claim 3, wherein the retainer includes a tube and the method further comprising: grasping proximal portion of implant with loop thread passing through to a proximal opening of the tube.

5. The method of claim 4, further comprising cutting one leg of the loop and stringing thread through tube to release the implant.

6. Method of claim 4, wherein said grasping is on two proximal corners of implant.

7. (canceled)

8. The method of claim 2, further including retracting the optics with said retracting of the sheath.

9. The method of claim 2, further comprising advancing said optics after said exposing.

10. The method of claim 9, wherein said advancing includes causing a distal portion of said optics to protrude from said distal opening of said lumen.

11. The method of claim 2, further comprising re-extending the sheath after said retracting to return the implant into the distal opening of the lumen.

12. A system for inserting an implant into a urethra comprising:
a sheath having a distal portion configured for insertion into the urethra while a proximal portion remains outside of the urethra
a retainer configured for retaining the implant in a distal portion of said sheath;
a connector connecting the implant to a distal portion of the retainer and a handle configured to facilitate exposure of the implant from the sheath and release of the connector.

13. The system of claim 12, wherein said retainer extends through a lumen of the sheath from the implant to a proximal opening of the lumen.

14. The system of claim 12, wherein said implant is retained in the sheath in a collapsed state.

15. The system of claim 14, wherein the implant is configured to expand automatically upon exposure from the sheath.

16. The system of claim 12, further comprising a retraction mechanism in said handle for retracting the sheath while the implant is retained in place by the retainer thereby exposing the implant in the urethra.

17. The system of claim 12, wherein the retainer includes a tube and the connector includes a thread.

18. The system of claim 17, wherein a loop of the thread passes out a distal end of the tube and is looped around the implant and wherein two free ends of the loop are exposed passing out a proximal opening of the tube.

19. The system of claim 18, wherein said handle is configured to facilitate cutting one of said free ends and stringing said thread back out of said retainer from said proximal opening of the tube thereby releasing said implant.

20. The system of claim 12, further comprising an optical assembly having a distal portion that fits through said sheath while said implant is retained in said distal portion and while said retainer and connector are affixed to the implant and the optical assembly having a proximal portion accessible from outside the sheath for viewing through a distal opening of the sheath.

21. The system of claim 12, wherein said handle further includes a mechanism for re-extending the sheath to return the implant into said distal portion of the sheath after said exposing.
